(19) United States
(12) Patent Application Publication
(10) Pub. No.: US 2026/0347842 A1
(43) Pub. Date: Nov. 5, 2026

(54) INTERFERING RNA FOR INHIBITING TUBB2 GENE EXPRESSION AND USE THEREOF

(71) Applicant: JENKEM TECHNOLOGY CO., LTD. (BEIJING), Beijing (CN)

(72) Inventors: Hongli JIA, Beijing (CN); Meina LIN, Beijing (CN); Mai YANG, Beijing (CN); Xiaomeng CHEN, Beijing (CN); Xuan ZHAO, Beijing (CN)

(21) Appl. No.: 19/114,765

(22) PCT Filed: Sep. 22, 2023
(86) PCT No.: PCT/CN2023/120769

§ 371 Date: Mar. 24, 2025

(30) Foreign Application Priority Data
Sep. 23, 2022 (CN) 202211167611.6

(51) Int. Cl. C12N 15/113; A61P 35/00

(52) U.S. Cl. CPC C12N 15/113; A61P 35/00; C12N 2310/14; C12N 2310/141; C12N 2310/531

(57) ABSTRACT

The present invention provides an interfering RNA targeting microtubule protein β2 (TUBB2). The interfering RNA can reduce the expression of TUBB2, thereby treating a disease related to the increase of TUBB2 expression or excessive proliferation of cells. The present invention further provides a lipid nanoparticle drug comprising the interfering RNA. The lipid nanoparticle drug is used for treating a disease related to the increase of TUBB2 expression or excessive proliferation of cells. The present invention further provides an antibody-nucleic acid conjugate drug, which comprises the interfering RNA and an antibody and is used for treating a disease related to the increase of TUBB2 expression or excessive proliferation of cells.

### TECHNICAL FIELD

The present disclosure relates to the technical field of molecular biology and biomedicine, and in particular to an interfering RNA, particularly an siRNA, for effectively inhibiting TUBB2 expression and use thereof.

### BACKGROUND

Tubulin is an essential component of the eukaryotic cytoskeleton, widely present in microtubules of living cells. Microtubules are fundamental elements of the eukaryotic cytoskeleton, primarily polymerized from α-tubulin and β-tubulin. Under normal conditions, a dynamic equilibrium exists between microtubules and tubulin dimers. Microtubules play a crucial role in cellular support and motility, helping cells resist compressive forces to maintain their intended shape and structure; additionally, microtubules are equally essential for processes such as cell movement, chromosome segregation during mitosis, and intracellular transport of organelles.

In humans, tubulin comprises five subgroups: α-tubulin, β-tubulin, γ-tubulin, δ- and ε-tubulin, and ζ-tubulin. β-Tubulin is of particular interest as it forms a type of microtubule expressed exclusively in neurons. Furthermore, all drugs binding to human tubulin also bind to β-tubulin, making it a therapeutic target.

Antimitotic chemotherapeutic drugs target tubulin, the principal protein in the mitotic spindle, and the composition of tubulin isoforms is considered a determinant for tumor progression diagnosis and cellular response to chemotherapy. This implies that the isoform composition differs between normal and tumor tissues, providing a theoretical basis for developing chemotherapeutic drugs against tumors. Currently, common anti-tubulin drugs include vinblastine, paclitaxel, and the like. Among these, paclitaxel can disrupt the dynamic equilibrium between microtubules and tubulin dimers, inducing and promoting tubulin polymerization while preventing depolymerization, thereby stabilizing microtubules. Vinblastine primarily inhibits tubulin polymerization, obstructing the formation of spindle microtubules and arresting nuclear division at metaphase; it may also induce nuclear collapse, manifesting as vacuolar pyknosis; by acting on the cell membrane, it interferes with amino acid transport across the cell membrane, inhibiting protein synthesis; through inhibition of RNA polymerase activity, it may inhibit RNA synthesis, eliminating cells during the G1 phase. These effects prevent the formation of spindles and spindle fibers during mitosis, inhibiting cell division and proliferation to exert antitumor effects. Thus, by inhibiting or promoting tubulin polymerization to disturb microtubule equilibrium, cell mitosis is affected, thereby inhibiting cell proliferation.

β2-Tubulin exists in the cell nucleus and can also exist in a non-microtubule form, significantly influencing cell behavior. β2-Tubulin participates in the development and progression of several tumor types, such as neuroepithelial tumors and brain tumors, colon cancer, and prostate cancer. In invasive metastatic tumors, nuclear-localized β2-tubulin has been identified, suggesting its potential interaction with anti-microtubule drugs like paclitaxel and vinblastine. β2-Tubulin not assembled into microtubules in the cytoplasm can interact with the voltage-dependent anion channel (VDAC) to provide energy for muscle cells. Studies reveal that β2-tubulin overexpression correlates with shorter life expectancy in colorectal cancer patients. Patients with tumors exhibiting nuclear-localized β2-tubulin have even shorter life expectancies.

Therefore, the present application has screened interfering RNA sequences with relatively high inhibitory activity, which have the potential to be developed into RNA drugs for tumor treatment. Additionally, siRNA molecules are relatively large and carry significant negative charges, making it difficult for them to pass through the negatively charged cell membrane. Therefore, identifying suitable delivery systems constitutes a critical aspect of siRNA drug development. The present application provides two feasible delivery approaches: lipid nanoparticles (LNP) and antibody-siRNA conjugates (ARC).

### SUMMARY

In a first aspect of the present disclosure, provided is an interfering RNA targeting a 02-tubulin (TUBB2) gene.

The interfering RNA inhibits TUBB2 expression.

A target site sequence of the interfering RNA comprises the nucleotide sequence set forth in any one or two or more of SEQ ID NOs: 1-13. Preferably, the target site sequence of the interfering RNA is set forth in any one of the nucleotide sequences of SEQ ID NOs: 1-13.

Preferably, the target site sequence of the interfering RNA comprises the nucleotide sequence set forth in any one or two or more of SEQ ID NOs: 1-10 or 12. Preferably, the target site sequence of the interfering RNA is set forth in any one of the nucleotide sequences of SEQ ID NOs: 1-10 or 12.

Further preferably, the target site sequence of the interfering RNA comprises the nucleotide sequence set forth in any one or two or more of SEQ ID NOs: 1-3 or 5-6 or 8-10. Preferably, the target site sequence of the interfering RNA is set forth in any one of the nucleotide sequences of SEQ ID NOs: 1-3 or 5-6 or 8-10.

In one specific embodiment of the present disclosure, the target site sequence of the interfering RNA comprises SEQ ID NOs: 2, 8, and/or 10. The interfering RNA comprises one or a combination of two or more of an siRNA, a dsRNA, an shRNA, an aiRNA, or an miRNA.

In one specific embodiment of the present disclosure, the interfering RNA is an siRNA.

The interfering RNA is 17-25 nt, preferably 19-21 nt, e.g., 17 nt, 18 nt, 19 nt, 20 nt, 21 nt, 22 nt, 23 nt, 24 nt, or 25 nt in length.

The interfering RNA further comprises an over-hang to increase the stability and activity of the interfering RNA.

Preferably, the interfering RNA comprises 1-10 over-hangs, further preferably, 2-4 over-hangs, e.g., 1, 2, 3, 4, 5, 6, 7, 8, 9, or 10 over-hangs.

The over-hang is located at the 3′ terminus of a sense strand and/or an antisense strand of the interfering RNA.

The over-hang is a deoxynucleoside; preferably, the over-hang may be n identical or different deoxynucleosides (e.g., deoxythymidine (dT), deoxycytidine (dC), and deoxyuridine (dU)), with n being an integer of 1-10 (e.g., 1, 2, 3, 4, 5, 6, 7, 8, 9, or 10).

In one specific embodiment of the present disclosure, the terminus of the sense strand and/or the antisense strand of the interfering RNA has 2 identical over-hangs.

In one specific embodiment of the present disclosure, the terminus of the sense strand and/or the antisense strand of the interfering RNA has 2 different over-hangs.

In one specific embodiment of the present disclosure, the over-hang is dTdT, dTdC, or dUdU.

In one specific embodiment of the present disclosure, the interfering RNA comprises a sense strand and/or an antisense strand.

Preferably, the sense strand and the antisense strand are a complementary pair.

Preferably, the sense strand comprises the nucleotide sequence set forth in any one or two or more of SEQ ID NOs: 14-26. Further preferably, the sense strand is set forth in any one of the nucleotide sequences of SEQ ID NOs: 14-26. Further preferably, the sense strand comprises the nucleotide sequence set forth in any one or two or more of SEQ ID NOs: 14-23 or 25. Still further preferably, the sense strand comprises the nucleotide sequence set forth in any one or two or more of SEQ ID NOs: 14-16 or 18-19 or 21-23.

In one specific embodiment of the present disclosure, the sense strand comprises SEQ ID NOs: 15, 21, and/23.

preferably, the antisense strand comprises the nucleotide sequence set forth in any one or two or more of SEQ ID NOs: 27-39. Further preferably, the antisense strand is set forth in any one of the nucleotide sequences of SEQ ID NOs: 27-39. Further preferably, the antisense strand comprises the nucleotide sequence set forth in any one or two or more of SEQ ID NOs: 27-36 or 38. Still further preferably, the antisense strand comprises the nucleotide sequence set forth in any one or two or more of SEQ ID NOs: 27-29 or 31-32 or 34-36.

In one specific embodiment of the present disclosure, the antisense strand comprises SEQ ID NOs: 28, 34, and/or 36.

In one specific embodiment of the present disclosure, the interfering RNA may further comprise at least one modification. The modified interfering RNA has better properties, such as higher stability and lower immune stimulation, compared with a corresponding unmodified interfering RNA.

The modification includes a modification on chemical structures of a base, a sugar ring, and/or a phosphate.

Preferably, the modification on a base includes, but is not limited to, a pyrimidine modification at position 5, a purine modification at position 8, and/or a 5-bromouracil substitution.

Preferably, the modification on a sugar ring includes, but is not limited to, a substitution of 2′-OH with H, OZ, Z, halo, SH, SZ, NH2, NHZ, NZ2, CN, or other groups, Z being an alkyl group.

The alkyl group refers to a hydrocarbyl that is linear or branched and that does not contain unsaturated bonds, and the hydrocarbyl is linked to the rest of the molecule via a single bond. A typical alkyl group contains 1-20 (e.g., 1, 2, 3, 4, 5, 6, 7, 8, 9, 10, 11, 12, 13, 14, 15, 16, 17, 18, 19, or 20) carbon atoms, such as methyl, ethyl, n-propyl, isopropyl, n-butyl, sec-butyl, isobutyl, tert-butyl, n-pentyl, isopentyl, neopentyl, tert-pentyl, n-hexyl, isohexyl, n-heptyl, isoheptyl, n-octyl, nonyl, decyl, undecyl, 1-methylundecyl, dodecyl, tridecyl, tetradecyl, pentadecyl, hexadecyl, heptadecyl, octadecyl, nonadecyl, and eicosyl. preferably, the modification on a phosphate backbone comprises, but is not limited to, a phosphorothioate modification.

Preferably, the modification further comprises nucleotides having inosine, queuosine, xanthine, 2′-methylribose, non-natural phosphodiester bond (e.g., methylphosphonate and phosphorothioate), and/or peptide.

In one specific embodiment of the present disclosure, the modification may be methylation, fluorination, a phosphorothioate modification, a pseudouridine modification, etc.

The modification may occur at any site in the sequence, e.g., at some sites or all sites.

The same sequence may have identical or different modifications at some sites or all sites.

In one specific embodiment of the present disclosure, the interfering RNA comprises one or a combination of two or more of the following combinations of the target site sequence, the sense strand sequence, and the antisense strand sequence:
A) SEQ ID NO: 1, SEQ ID NO: 14, SEQ ID NO: 27;
B) SEQ ID NO: 2, SEQ ID NO: 15, SEQ ID NO: 28;
C) SEQ ID NO: 3, SEQ ID NO: 16, SEQ ID NO: 29;
D) SEQ ID NO: 5, SEQ ID NO: 18, SEQ ID NO: 31;
E) SEQ ID NO: 6, SEQ ID NO: 19, SEQ ID NO: 32;
F) SEQ ID NO: 8, SEQ ID NO: 21, SEQ ID NO: 34;
G) SEQ ID NO: 9, SEQ ID NO: 22, SEQ ID NO: 35;
H) SEQ ID NO: 10, SEQ ID NO: 23, SEQ ID NO: 36;
I) SEQ ID NO: 8, SEQ ID NO: 56, SEQ ID NO: 60;
J) SEQ ID NO: 8, SEQ ID NO: 57, SEQ ID NO: 61;
K) SEQ ID NO: 10, SEQ ID NO: 58, SEQ ID NO: 62;
L) SEQ ID NO: 10, SEQ ID NO: 59, SEQ ID NO: 63.

Preferably, the interfering RNA comprises combination B), combination F), and/or combination H).

In one specific embodiment of the present disclosure, the interfering RNA comprises one or two or more of the nucleic acid sequences shown in Table 1 or Table 15.

The interfering RNA can be prepared and obtained by any method in the prior art, such as chemical synthesis.

In a second aspect of the present disclosure, provided is a delivery system comprising the interfering RNA described above.

Preferably, the delivery system further comprises a vector.

Preferably, the vector can be any vector suitable for delivering the interfering RNA of the present disclosure described above to a target tissue or a target cell, such as those disclosed in the prior art (e.g., Chen Zhonghua, Zhu Desheng, Li Jun, Huang Zhanqin. “Advances in non-viral vector research of siRNA”. Chinese Pharmacological Bulletin. 2015, 31 (7): 910-4; Wang Rui, Qu Bingnan, Yang Jing. “Advances in research of siRNA-carrying nano preparation”. China Pharmacy 2017, 28 (31): 4452-4455).

In one specific embodiment of the present disclosure, the vector is a viral vector; preferably, the viral vector includes, but is not limited to, one or two or more of a lentivirus vector, a retrovirus vector, an adenovirus vector, an adeno-associated virus vector, a poxvirus vector, a herpesvirus vector, etc.

In one specific embodiment of the present disclosure, the vector is a non-viral vector; preferably, the non-viral vector includes, but is not limited to, any one or a combination of two or more of a liposome, a lipid nanoparticle (LNP), a polymer, a polypeptide, an antibody, an aptamer, or N-acetylgalactosamine (GalNAc); further preferably, the non-viral vector includes a lipid nanoparticle (LNP). A weight ratio of the interfering RNA to the non-viral vector can be any value in the range of 1:1 to 1:50 (e.g., 1:1, 1:5, 1:6, 1:7, 1:8, 1:9, 1:10, 1:15, 1:20, 1:25, 1:30, 1:35, 1:40, 1:45, or 1:50), preferably any value in the range of 1:1 to 1:10.

Preferably, the lipid nanoparticle/liposome described above includes one or a combination of two or more of a cationic lipid, a neutral lipid, a polyethylene glycol lipid, a steroidal lipid, or an anionic lipid.

Further preferably, the cationic lipid includes one or a combination of two or more of stearamide (SA), lauryltrimethylammonium bromide, hexadecyltrimethylammonium bromide, myristyltrimethylammonium bromide, dimethyldioctadecylammonium bromide (DDAB), [(4-hydroxybutyl)azanediyl]bis(hexane-6,1-diyl)bis(2-hexyldecanoate) (ALC-0315), 1,2-dioleoyloxy-3-(trimethylammonium)propane (DOTAP), 1,2-di-(9Z-octadecenoyl)-3-trimethylammonium-propane and 1,2-dihexadecanoyl-3-trimethylammonium-propane, 30-[N—(N′,N′-dimethylaminoethane)-carbamoyl]cholesterol (DC cholesterol), dimethyldioctadecylammonium (DDA), 1,2-dimyristoyl-3-trimethylammonium propane (DMTAP), dipalmitoyl(C16:0)trimethylammonium propane (DPTAP), distearoyltrimethylammonium propane (DSTAP), N-[1-(2,3-diallyloxy)propyl]-N,N,N-trimethylammonium chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium propane (DODAP), 1,2-dilinoleyloxy-3-dimethylaminopropane (DLinDMA), 1,2-ditetradecanoyl-3-dimethylammonium-propane, 1,2-dihexadecanoyl-3-dimethylammonium-propane and 1,2-dioctadecanoyl-3-dimethylammonium-propane, 1,2-dioleoyl-c-(4′-trimethylammonium)-butanoyl-sn-glycerol (DOTB), dioctadecylamidoalanyl spermine, SAINT-2, polycationic lipid 2,3-dioleoyloxy-N-[2(spermine-carboxamido)ethyl]-N,N-dimethyl-1-propanaminium trifluoroacetate (DOSPA),

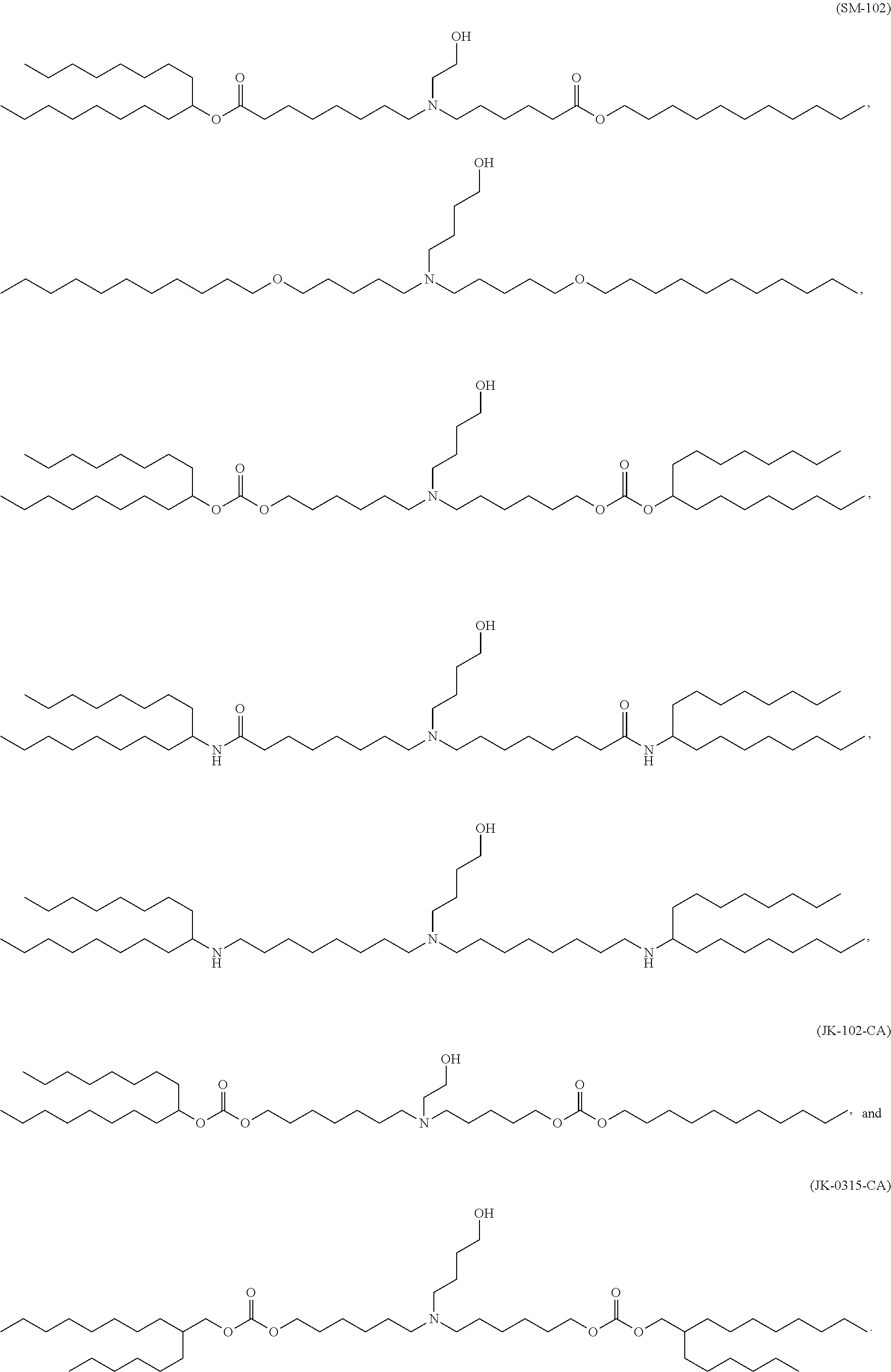

Preferably, the cationic lipid is a steroid-cationic lipid compound,

and the compound has a structure of one or two or more of

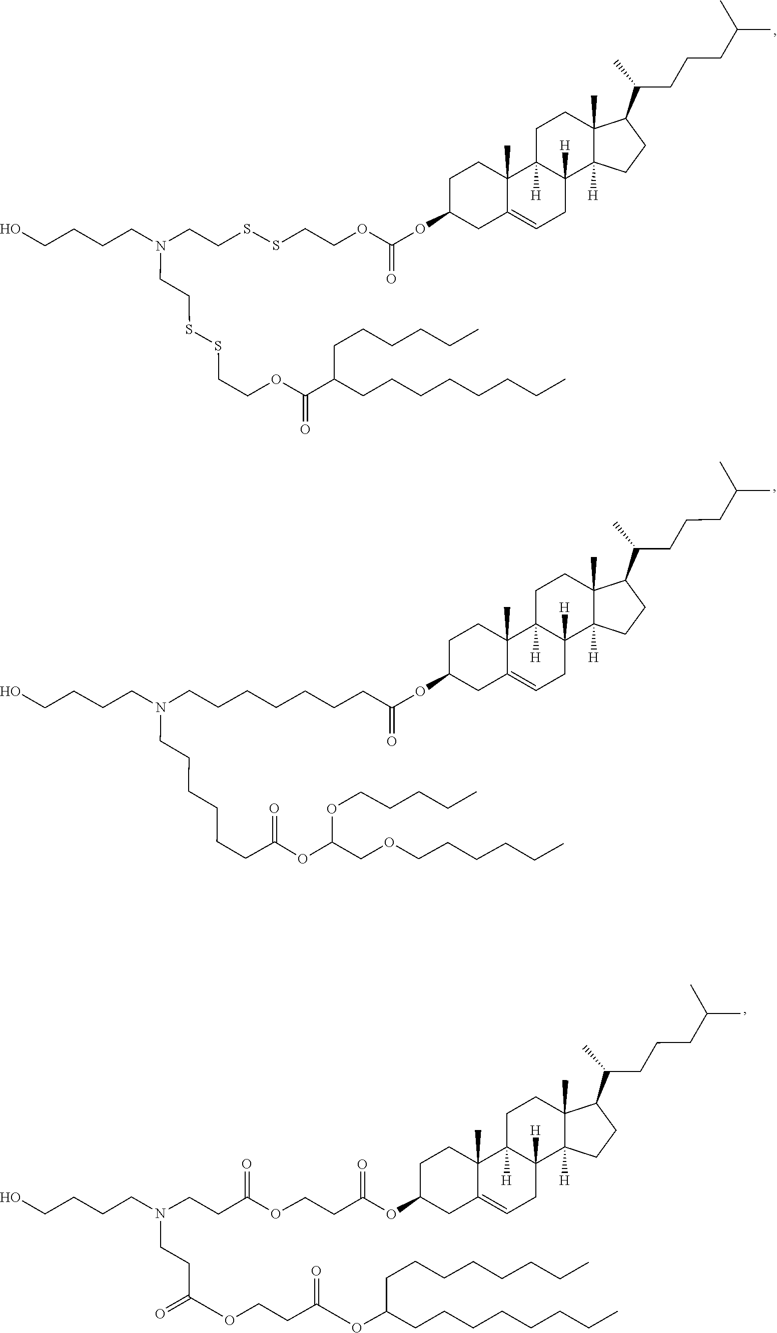

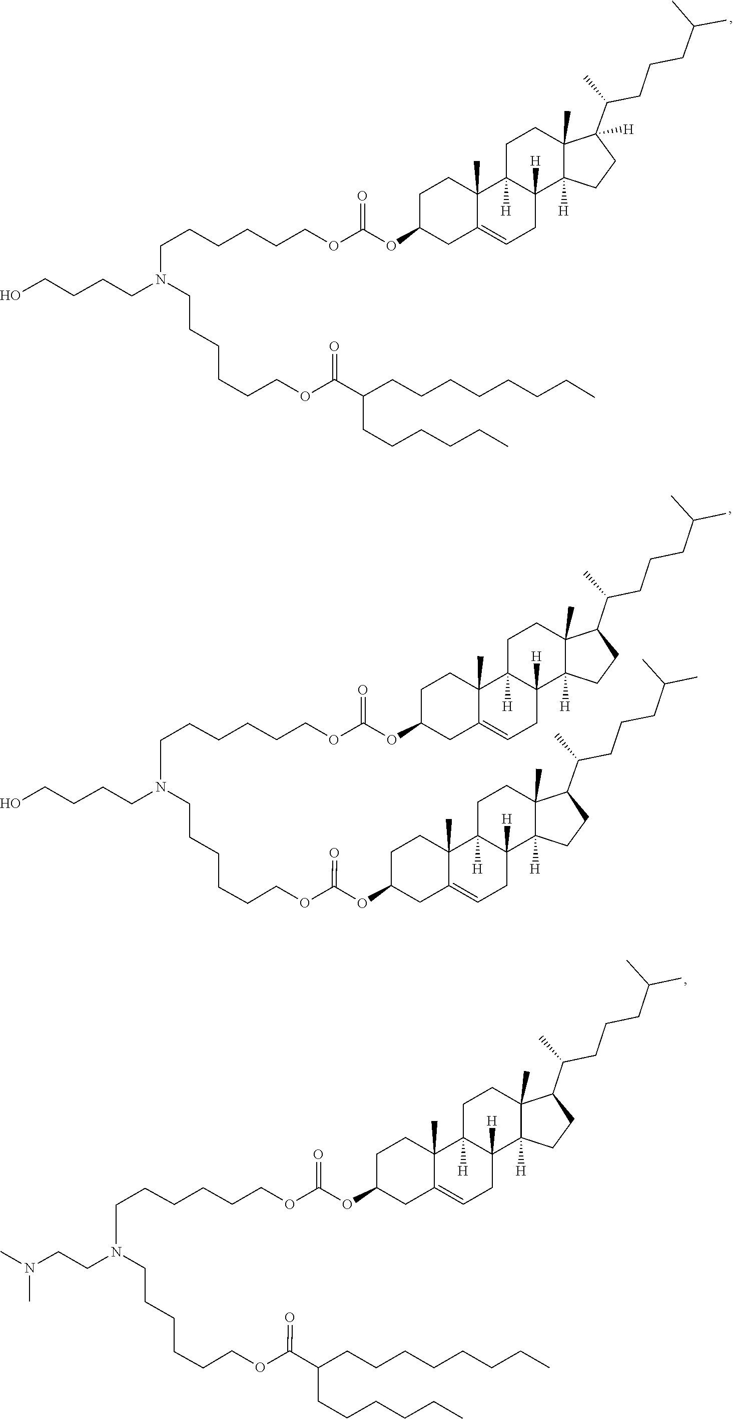

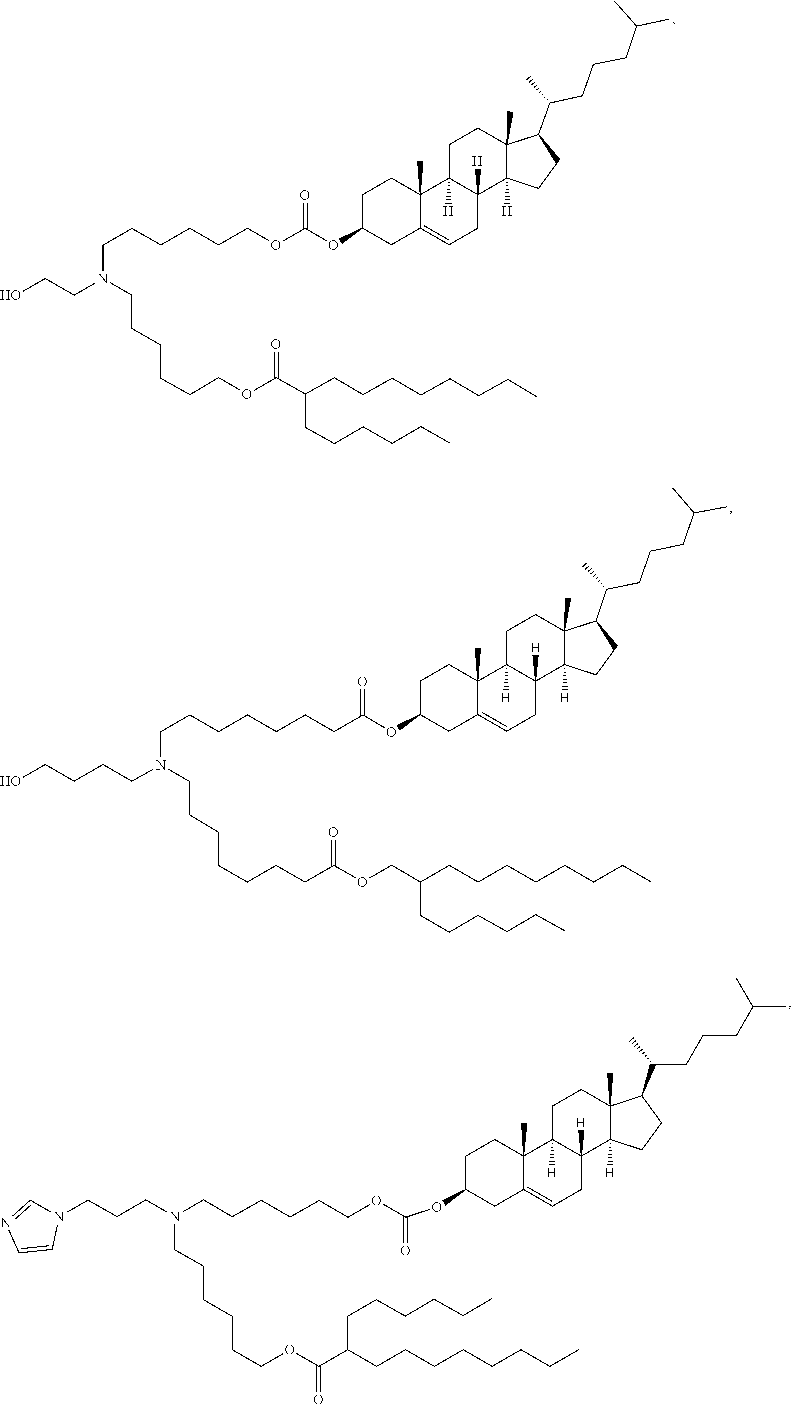

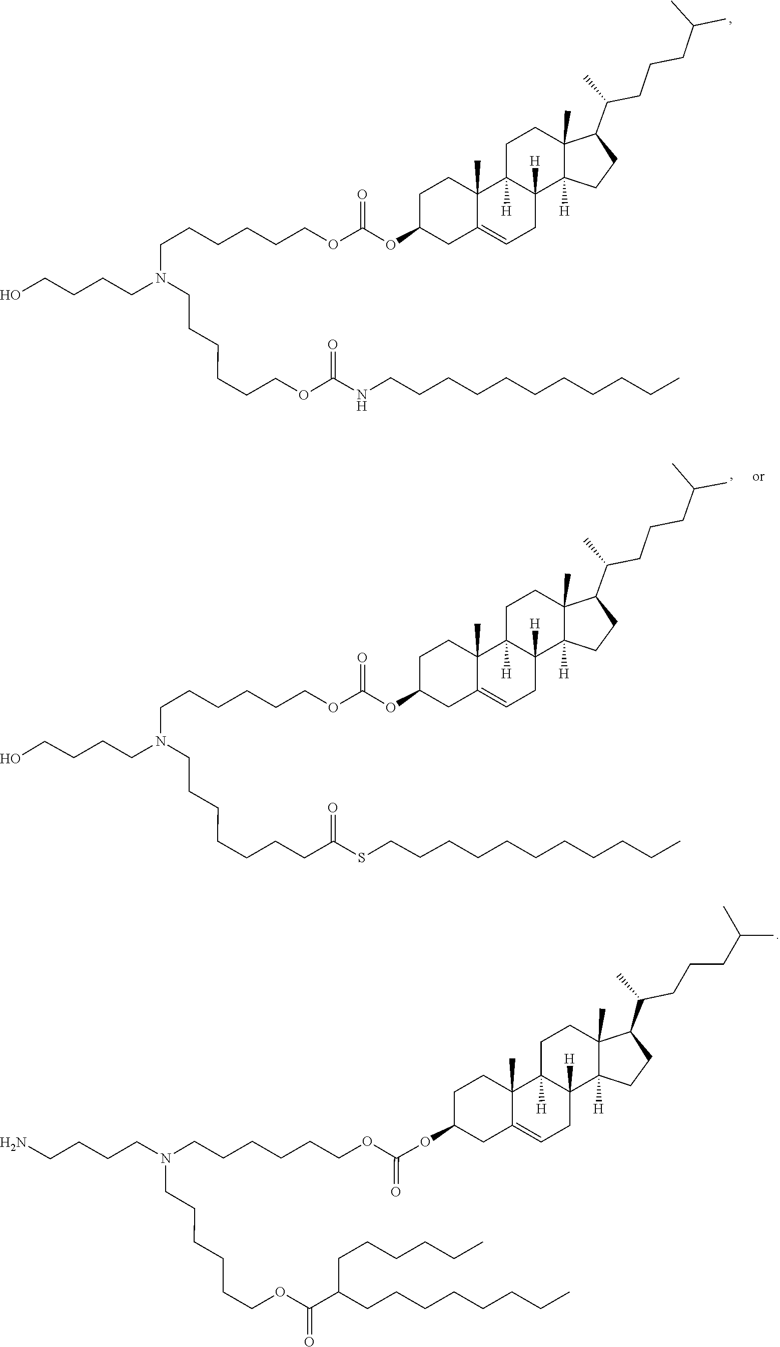

Further preferably, the neutral lipid includes one or a combination of two or more of 1,2-distearoyl-sn-glycero-3-phosphocholine (DSPC), 1,2-dipalmitoyl-sn-glycero-3-phosphocholine (DPPC), 1,2-dioleoyl-sn-glycero-3-phosphoethanolamine (DOPE), 1,2-dipalmitoyl-sn-glycero-3-phosphoethanolamine (DPPE), 1,2-dimyristoyl-sn-glycero-3-phosphoethanolamine (DMPE), 2-dioleoyl-sn-glycero-3-phospho-(1′-rac-glycerol) (DOPG), oleoyl phosphatidylcholine (POPC), 1-palmitoyl-2-oleoyl phosphatidylethanolamine (POPE), or distearoylphosphatidylethanolamine (DSPE).

Further preferably, the polyethylene glycol lipid includes one or a combination of two or more of 2-[(polyethylene glycol)-2000]-N,N-ditetradecylacetamide (ALC-0159), 1,2-dimyristoyl-sn-glyceromethoxypolyethylene glycol (PEG-DMG), 1,2-distearoyl-sn-glycero-3-phosphoethanolamine-N-[amino(polyethylene glycol)](PEG-DSPE), PEG-disterol glycerol (PEG-DSG), PEG-dipalmitoyl, PEG-dioleyl, PEG-distearyl, PEG-diacylglycerol amide (PEG-DAG), PEG-dipalmitoyl phosphatidylethanolamine (PEG-DPPE) or PEG-1,2-dimyristoylacyloxypropyl-3-amine (PEG-c-DMA),

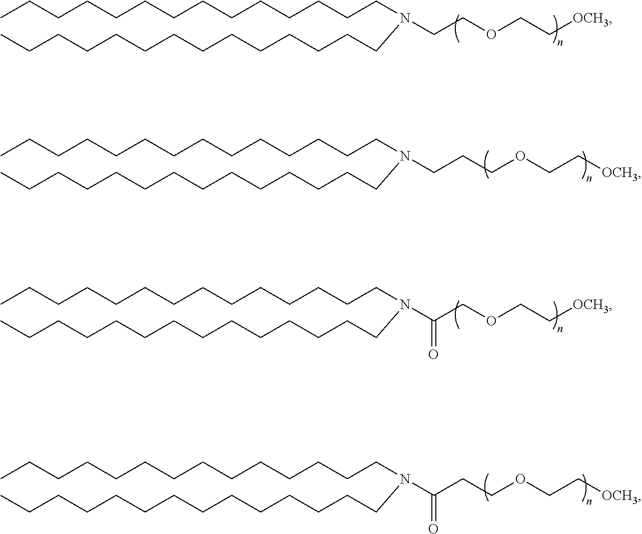

etc.,

wherein n is selected from integers of 20-300, e.g., 20, 30, 50, 80, 100, 150, 200, 250, and 300.

Preferably, the polyethylene glycol lipid is a polyethylene glycol lipid with a single molecular weight; preferably, the polyethylene glycol lipid includes

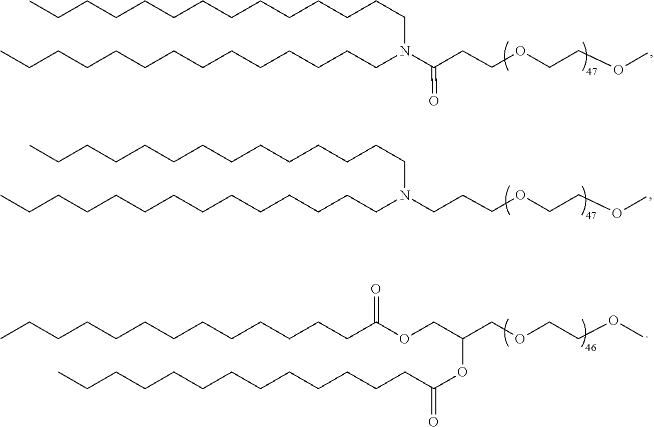

Further preferably, the anionic liposome includes dioleoyl phosphatidylglycerol and/or dioleoyl phosphatidylethanolamine, etc.

Further preferably, the steroidal lipid includes one or a combination of two or more of avenasterol, β-sitosterol, brassicasterol, ergocalciferol, campesterol, cholestanol, cholesterol, coprostanol, dehydrocholesterol, desmosterol, dihydroergocalciferol, dihydrocholesterol, dihydroergosterol, dinosterol, epicholesterol, ergosterol, fucosterol, hexahydrolumisterol, hydroxycholesterol, lanosterol, lumisterol, saringosterol, sitostanol, sitosterol, stigmastanol, stigmasterol, cholic acid, glycocholic acid, taurocholic acid, deoxycholic acid, or lithocholic acid.

Preferably, the polymer described above may be a synthetic polymer (e.g., polyethyleneimine and cyclodextrin) or a natural polymer (e.g., chitosan and atelocollagen) or a mixture thereof.

Preferably, the polypeptide described above may be a cell penetrating peptide (CPP) (e.g., protamine, Tat peptide, transportan peptide, penetratin peptide, and oligo-arginine peptide).

Preferably, the antibody described above may be a single chain antibody (e.g., scFv-tp and scFv-9R).

The delivery system of the present application can also encapsulate various components beneficial to a human body, and directly deliver the components into cells to exert their effects, thereby achieving the desired results more quickly and effectively.

In a third aspect of the present disclosure, provided is a cell comprising the interfering RNA described above or the delivery system described above.

The expression level of TUBB2 in the cell is inhibited.

The cell may be a tumor cell.

In a fourth aspect of the present disclosure, provided is a preparation method for a cell, which comprises introducing into a cell the interfering RNA described above or the delivery system described above.

In a fifth aspect of the present disclosure, provided is a lipid nanoparticle comprising the interfering RNA described above.

Preferably, the lipid nanoparticle further comprises one or two or more of a polyethylene glycol lipid compound, a cationic lipid, a steroidal lipid, or a neutral lipid.

The polyethylene glycol lipid compound, the cationic lipid, the steroidal lipid, and the neutral lipid are as defined in the second aspect of the present application.

In one specific embodiment of the present disclosure, the lipid nanoparticle comprises the interfering RNA described above, as well as a polyethylene glycol lipid compound, a cationic lipid (excluding a steroid-cationic lipid compound), a steroidal lipid, and a neutral lipid.

Preferably, in the lipid nanoparticle, a molar ratio of the polyethylene glycol lipid compound to the cationic lipid to the steroidal lipid to the neutral lipid is (0.5-5):(30-55):(30-55):(5-20), e.g., (0.5, 1, 2, 3, 4, or 5):(30, 31, 32, 33, 34, 35, 36, 37, 38, 39, 40, 41, 42, 43, 44, 45, 46, 47, 48, 49, 50, 51, 52, 53, 54, or 55):(30, 31, 32, 33, 34, 35, 36, 37, 38, 39, 40, 41, 42, 43, 44, 45, 46, 47, 48, 49, 50, 51, 52, 53, 54, or 55):(5, 6, 7, 8, 9, 10, 11, 12, 13, 14, 15, 16, 17, 18, 19, or 20). Preferably, a molar ratio of the polyethylene glycol lipid compound, the cationic lipid, the steroidal lipid, and the neutral lipid is (1-5):(35-50):(40-50):(8-15).

In one specific embodiment of the present disclosure, the lipid nanoparticle comprises the interfering RNA described above, as well as a steroid-cationic lipid compound, a second lipid, and a polyethylene glycol lipid. The second lipid is selected from a neutral lipid, a zwitterionic lipid, and an anionic lipid.

Preferably, in the lipid nanoparticle, the molar ratio of the steroid-cationic lipid to the second lipid to the polyethylene glycol lipid is (10-30):(60-80):(10-25), e.g., (10, 15, 20, 25, or 30):(60, 65, 70, 75, or 80):(10, 15, 20, or 25), preferably (20-30):(60-70):(10-20).

The lipid nanoparticle of the present application can also encapsulate various components beneficial to a human body, and directly deliver the components into cells to exert their effects, thereby achieving the desired results more quickly and effectively.

The lipid nanoparticle can be prepared by using a conventional preparation method for preparing a lipid nanoparticle in the art, for example, high pressure homogenization, emulsion precipitation, ultrasonic dispersion, etc.

In a sixth aspect of the present disclosure, provided is a medicament or kit, which comprises the interfering RNA described above, the delivery system described above, the lipid nanoparticle described above, or the cell described above.

The medicament further comprises a pharmaceutically acceptable auxiliary material.

Preferably, the pharmaceutically acceptable auxiliary material includes, but is not limited to, a carrier, a diluent, a binder, a lubricant, a wetting agent, etc.

Preferably, an administration mode of the medicament includes, but is not limited to, oral administration, intestinal administration, subcutaneous injection, intramuscular injection, intravenous injection, nasal administration, transdermal administration, subconjunctival administration, intraocular administration, orbital administration, retrobulbar administration, retinal administration, choroidal administration, intrathecal injection, etc.

Preferably, the dosage form of the medicament includes, but is not limited to, a tablet, a capsule, a pill, an injection, an inhalant, a troche, a suppository, an emulsion, a microemulsion, a submicroemulsion, a nanoparticle, a gel, a powder, a suspoemulsion, a cream, a jelly, a spray, etc. The various dosage forms of the medicament can be prepared according to conventional production methods in the field of pharmaceuticals.

Preferably, a mass content of the interfering RNA described above, the delivery system described above, or the cell described above in the medicament may be 1%-100%, e.g., 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, 99.5%, or 100%.

In a seventh aspect of the present disclosure, provided is an antibody-nucleic acid drug conjugate, which comprises the interfering RNA described above or the delivery system described above or the lipid nanoparticle described above.

The antibody-nucleic acid drug conjugate comprises one or more interfering RNAs, as well as an antibody.

In the antibody-nucleic acid drug conjugate, the antibody and the nucleic acid may be directly linked or linked via a linking group or a linker peptide.

The antibody-nucleic acid drug conjugate has a general formula (I):

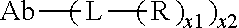

wherein R is the interfering RNA described above;
x1 is an integer of 1-144; preferably, x1 is an integer of 1-9; more preferably, x1 is an integer of 1-3;
x2 is an integer of 1-8; preferably, x2 is an integer of 1-2;
Ab is an antibody, a protein, or a polypeptide; and
L is a linking unit connecting Ab and R.

Preferably, the moiety L has a structure of general formula (II):

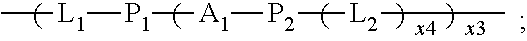

wherein
x3 in formula II is selected from integers of 1-12, preferably 1-3;
x4 in formula II is selected from integers of 1-12, preferably 1-3;
P₁ and P₂ are identical or different polyethylene glycol residues;
L₁ is a linking unit connecting Ab and P₁;
L₂ is a linking unit connecting P₂ and R;
A₁ is a linking unit connecting P₁ and P₂;
preferably, P₁ and P₂ are independently selected from linear, Y-shaped, and multi-branched polyethylene glycol residues;
preferably, when P₁ and P₂ are polyethylene glycol with a single molecular weight, the molecular weight is 88-4400 Da, and more preferably, the molecular weight of P₁ and P₂ is 176-1056 Da; preferably, when P₁ and P₂ are polyethylene glycol with a non-single molecular weight, the molecular weight is 1000 Da-40 kDa; and
more preferably, the molecular weight of P₁ and P₂ is 2000 Da-10 kDa.

Preferably, L₁ is a linking group selected from one or a combination of two or more of linear or branched C₁₋₁₂ alkylene, C₆₋₁₂ arylene, C₃₋₁₂ cycloalkylene, —S—,

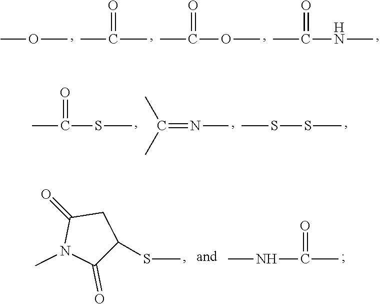

an optional H atom on the linear or branched C₁₋₁₂ chain alkylene, C₆₋₁₂ arylene, or C₃₋₁₂ cycloalkylene is substituted with one or a combination of two or more of —H, —F, —Cl, —Br, —I, —O—, —S—, —SO₂, —NO₂, C₁₋₁₂ chain alkyl, C₃₋₁₂ cycloalkyl, C₆₋₁₂ aralkyl, substituted or unsubstituted heterocyclyl, substituted or unsubstituted heterocyclylalkyl,

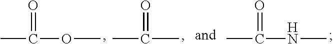

preferably, L₂ is a linking group selected from one or a combination of two or more of linear or branched C₁₋₁₂ alkylene, C₆₋₁₂ arylene, C₃₋₁₂ cycloalkylene, —S—,

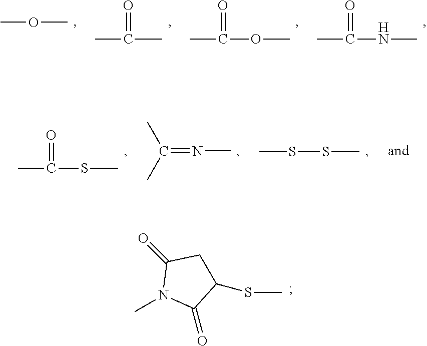

an optional H atom on the linear or branched C₁₋₁₂ chain alkylene, C₆₋₁₂ arylene, or C₃₋₁₂ cycloalkylene is substituted with a combination of one or more of —H, —F, —Cl, —Br, —I, —O—, —S—, —SO₂, —NO₂, C₁₋₁₂ chain alkyl, C₃₋₁₂ cycloalkyl, C₆₋₁₂ aralkyl, substituted or unsubstituted heterocyclyl, substituted or unsubstituted heterocyclylalkyl,

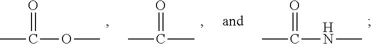

preferably, L₁ is an amide bond, a hydrazone bond, or a thiol-maleimide bond;
more preferably, L₁ is an amide bond;
preferably, L₂ is a disulfide bond or a thiol-maleimide bond; and
more preferably, L₂ is a disulfide bond.

Preferably, A₁ is a linking group connecting P₁ and P₂, and is selected from one or a combination of two or more of linear or branched C₁₋₁₂ alkylene, C₆₋₁₂ arylene, C₃₋₁₂ cycloalkylene, —S—,

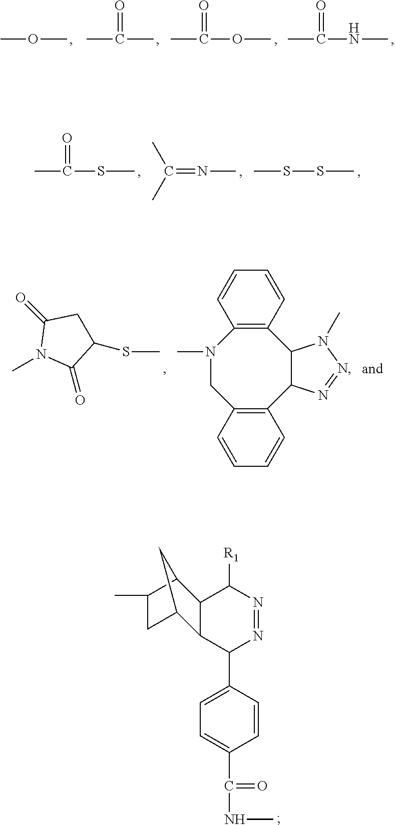

an optional H atom on the linear or branched C₁₋₁₂ chain alkylene, C₆₋₁₂ arylene, or C₃₋₁₂ cycloalkylene is substituted with one or a combination of two or more of —H, —F, —Cl, —Br, —I, —O—, —S—, —SO₂, —NO₂, C₁₋₁₂ chain alkyl, C₃₋₁₂ cycloalkyl, C₆₋₁₂ aralkyl, substituted or unsubstituted heterocyclyl, substituted or unsubstituted heterocyclylalkyl,

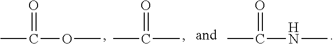

Further preferably, the antibody-nucleic acid drug conjugate has a general formula (IV):

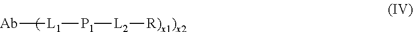

wherein L₁ is a linking group selected from one or a combination of two or more of linear or branched C₁₋₁₂ alkylene, C₆₋₁₂ arylene, C₃₋₁₂ cycloalkylene, —S—,

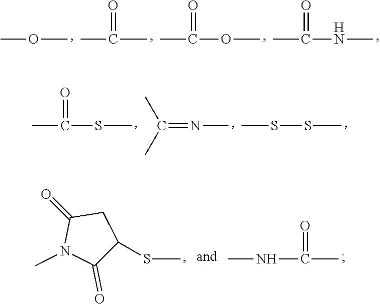

preferably, P₁ are identical or different polyethylene glycol residues;
preferably, when P₁ is polyethylene glycol with a single molecular weight, the molecular weight is 88-4400 Da; more preferably, the molecular weight of P₁ is 176-1056 Da;
preferably, when P₁ is a polyethylene glycol with a non-single molecular weight, the molecular weight is 1000 Da-40 kDa; more preferably, the molecular weight of P₁ is 2000 Da-10 kDa.
L₂ is a linking group selected from one or a combination of two or more of linear or branched C₁₋₁₂ alkylene, C₆₋₁₂ arylene, C₃₋₁₂ cycloalkylene, —S—,

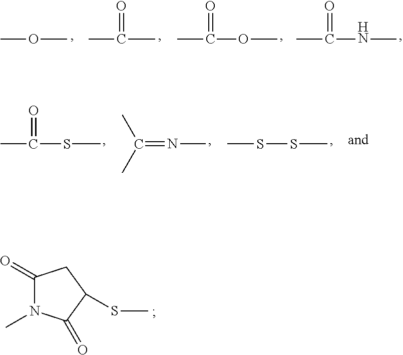

x1 is an integer of 1-144; preferably, x1 is an integer of 1-9; more preferably, x1 is an integer of 1-3;
x2 is an integer of 1-8; preferably, x2 is an integer of 1-2.

In one specific embodiment of the present disclosure, the antibody-nucleic acid drug conjugate has the following structure:

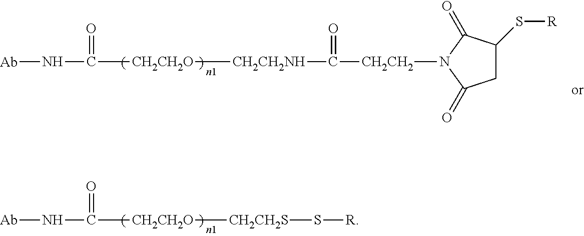

wherein n₁ is selected from integers of 4-100, preferably 4-24; n1 can be a constant value or an average value.

In one specific embodiment of the present disclosure, the antibody-nucleic acid drug conjugate has the following structure:

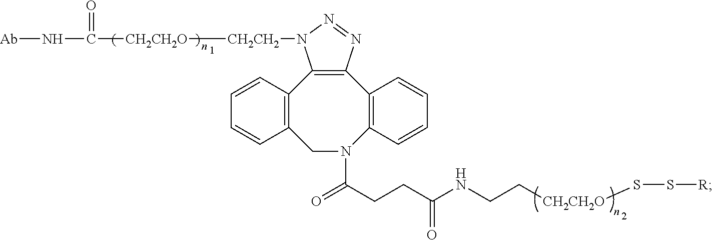

n₁ and n₂ are independently selected from integers of 4-100, preferably 4-24. n1 and n2 may be constant values or average values.

Preferably, Ab is selected from a monoclonal antibody, a polyclonal antibody, an antibody fragment, and an antibody fusion fragment.

The antibody may be a single domain antibody or a single chain antibody.

Further preferably, Ab is a monoclonal antibody; more preferably, the monoclonal antibody is reactive to an antigen or an epitope thereof associated with cancer, a malignant cell, an infectious organism, or an autoimmune disease.

In one specific embodiment of the present disclosure, Ab is selected from an anti-HER2 antibody, an anti-EGFR antibody, an anti-PMSA antibody, an anti-VEGFR antibody, an anti-CD30 antibody, an anti-CD22 antibody, an anti-CD56 antibody, an anti-CD29 antibody, an anti-GPNMB antibody, an anti-CD138 antibody, an anti-CD74 antibody, an anti-ENPP3 antibody, an anti-Nectin-4 antibody, an anti-EGFRVIII antibody, an anti-SLC44A4 antibody, an anti-mesothelin antibody, an anti-ET8R antibody, an anti-CD37 antibody, an anti-CEACAM5 antibody, an anti-CD70 antibody, an anti-MUC16 antibody, an anti-CD79b antibody, an anti-MUC16 antibody, an anti-Muel antibody, an anti-CD3 antibody, an anti-CD28 antibody, an anti-CD38 antibody, an anti-CD19 antibody, an anti-PD-L1 antibody, an anti-4-1BB antibody, etc.

In an eighth aspect of the present disclosure, provided is use of the interfering RNA described above, the delivery system described above, the cell described above, the medicament or kit described above, the lipid nanoparticle described above, or the antibody-nucleic acid drug conjugate described above, which comprises
A) use in preparing a medicament for treating and/or preventing a disease related to an increase of TUBB2 expression or excessive proliferation of cells;
B) use in inhibiting TUBB2 expression; or
C) use in treating and/or preventing a disease related to an increase of TUBB2 expression or excessive proliferation of cells.

Preferably, a working concentration of the interfering RNA, the delivery system described above, the cell described above, the medicament or kit described above, the lipid nanoparticle described above, or the antibody-nucleic acid drug conjugate described above used in the treatment is 0.01-1000 nM, e.g., 0.01 nM, 1 nM, 2 nM, 3 nM, 4 nM, 5 nM, 6 nM, 7 nM, 8 nM, 9 nM, 10 nM, 15 nM, 20 nM, 25 nM, 30 nM, 35 nM, 40 nM, 45 nM, 50 nM, 55 nM, 60 nM, 65 nM, 70 nM, 75 nM, 80 nM, 85 nM, 90 nM, 95 nM, 100 nM, 200 nM, 300 nM, 400 nM, 500 nM, 600 nM, 700 nM, 800 nM, 900 nM, or 1000 nM.
wherein preferably, the disease related to an increase of TUBB2 expression or excessive proliferation of cells comprises a tumor. Further preferably, the tumor includes a gastrointestinal tumor (oral cancer, tongue cancer, esophageal cancer, gastric cancer, liver cancer, pancreatic cancer, colorectal cancer, etc.), a nervous system tumor (glioma (e.g., neuroglioma), a neuroepithelial tumor, schwannoma, astrocytoma, neurofibroma (e.g., neurofibrosarcoma), ependymoma, medulloblastoma, meningioma, brain metastasis, etc.), a respiratory tract tumor (nasopharyngeal carcinoma, laryngeal carcinoma, bronchial carcinoma, lung cancer, etc.), a urogenital neoplasm (prostate cancer, a kidney tumor, bladder cancer, breast cancer, cervical cancer, ovarian cancer, placental choriocarcinoma, a urinary tract system tumor, a seminal vesicle tumor, a testicular and paratesticular tumor, a penile tumor, vaginal cancer, vulvar cancer, uterine cancer, endometrial cancer, etc.), a hematological and lymphatic system tumor (leukemia (e.g., chronic myelocytic leukemia, acute myeloid leukemia, chronic lymphocytic leukemia, myelodysplastic syndrome, chronic myelocytic leukemia, acute lymphocytic leukemia, etc.), multiple myeloma, mesothelioma, myeloma, and lymphoma (e.g., non-Hodgkin lymphoma, Hodgkin lymphoma, cutaneous T-cell lymphoma), a cutaneous system tumor (e.g., skin cancer, epidermoid cancer, melanoma, basal cell carcinoma, squamous cell carcinoma, Paget's disease, Kaposi's sarcoma, Merkel cell carcinoma, atypical fibroxanthoma, an adnexal tumor, cutaneous T-cell lymphoma (mycosis fungoides), etc.), etc., as well as rhabdomyosarcoma, head and neck cancer, etc.

Further preferably, the tumor is selected from a urogenital neoplasm and/or a cutaneous system tumor.

In one specific embodiment of the present disclosure, the tumor is selected from prostate cancer, breast cancer, and melanoma.

In a ninth aspect of the present disclosure, provided is a method for inhibiting TUBB2 expression, which comprises adding the interfering RNA described above, the delivery system described above, the cell described above, the lipid nanoparticle described above, the medicament or kit described above, or the antibody-nucleic acid drug conjugate described above.

Preferably, the method inhibits TUBB2 expression in a cell, and the cell is a tumor cell.

Preferably, the method comprises delivering into a cell the interfering RNA described above. The delivery is performed using the delivery system described above.

In a tenth aspect of the present disclosure, provided is a method for treating a disease related to an increase of TUBB2 expression or excessive proliferation of cells, which comprises administering to a subject a therapeutically effective amount of the interfering RNA described above, the delivery system described above, the lipid nanoparticle described above, the cell described above, the medicament or kit described above, or the antibody-nucleic acid drug conjugate described above.
wherein preferably, the disease related to an increase of TUBB2 expression or excessive proliferation of cells comprises a tumor. Further preferably,

The tumor includes a gastrointestinal tumor (oral cancer, tongue cancer, esophageal cancer, gastric cancer, liver cancer, pancreatic cancer, colorectal cancer, etc.), a nervous system tumor (glioma (e.g., neuroglioma), a neuroepithelial tumor, schwannoma, astrocytoma, neurofibroma (e.g., neurofibrosarcoma), ependymoma, medulloblastoma, meningioma, brain metastasis, etc.), a respiratory tract tumor (nasopharyngeal carcinoma, laryngeal carcinoma, bronchial carcinoma, lung cancer, etc.), a urogenital neoplasm (prostate cancer, a kidney tumor, bladder cancer, breast cancer, cervical cancer, ovarian cancer, placental choriocarcinoma, a urinary tract system tumor, a seminal vesicle tumor, a testicular and paratesticular tumor, a penile tumor, vulvar cancer, vaginal cancer, uterine cancer, endometrial cancer, etc.), a hematological and lymphatic system tumor (leukemia (e.g., chronic myelocytic leukemia, acute myeloid leukemia, chronic lymphocytic leukemia, myelodysplastic syndrome, chronic myelocytic leukemia, acute lymphocytic leukemia, etc.), multiple myeloma, mesothelioma, myeloma, and lymphoma (e.g., non-Hodgkin lymphoma, Hodgkin lymphoma, cutaneous T-cell lymphoma), a cutaneous system tumor (e.g., skin cancer, epidermoid cancer, melanoma, basal cell carcinoma, squamous cell carcinoma, Paget's disease, Kaposi's sarcoma, Merkel cell carcinoma, atypical fibroxanthoma, an adnexal tumor, cutaneous T-cell lymphoma (mycosis fungoides), etc.), etc., as well as rhabdomyosarcoma, head and neck cancer, etc.

In an eleventh aspect of the present disclosure, provided is a method for treating a tumor, which comprises administering to a subject a therapeutically effective amount of the interfering RNA described above, the delivery system described above, the cell described above, the lipid nanoparticle described above, the medicament or kit described above, or the antibody-nucleic acid drug conjugate described above.

Preferably, the tumor includes a gastrointestinal tumor (oral cancer, tongue cancer, esophageal cancer, gastric cancer, liver cancer, pancreatic cancer, colorectal cancer, etc.), a nervous system tumor (glioma (e.g., neuroglioma), a neuroepithelial tumor, schwannoma, astrocytoma, neurofibroma (e.g., neurofibrosarcoma), ependymoma, medulloblastoma, meningioma, brain metastasis, etc.), a respiratory tract tumor (nasopharyngeal carcinoma, laryngeal carcinoma, bronchial carcinoma, lung cancer, etc.), a urogenital neoplasm (prostate cancer, a kidney tumor, bladder cancer, breast cancer, cervical cancer, ovarian cancer, placental choriocarcinoma, a urinary tract system tumor, a seminal vesicle tumor, a testicular and paratesticular tumor, a penile tumor, vulvar cancer, vaginal cancer, uterine cancer, endometrial cancer, etc.), a hematological and lymphatic system tumor (leukemia (e.g., chronic myelocytic leukemia, acute myeloid leukemia, chronic lymphocytic leukemia, myelodysplastic syndrome, chronic myelocytic leukemia, acute lymphocytic leukemia, etc.), multiple myeloma, mesothelioma, myeloma, and lymphoma (e.g., non-Hodgkin lymphoma, Hodgkin lymphoma, cutaneous T-cell lymphoma), a cutaneous system tumor (e.g., skin cancer, epidermoid cancer, melanoma, basal cell carcinoma, squamous cell carcinoma, Paget's disease, Kaposi's sarcoma, Merkel cell carcinoma, atypical fibroxanthoma, an adnexal tumor, cutaneous T-cell lymphoma (mycosis fungoides), etc.), etc., as well as rhabdomyosarcoma, head and neck cancer, etc.

Further preferably, the tumor is selected from a urogenital neoplasm and/or a cutaneous system tumor.

In one specific embodiment of the present disclosure, the tumor is selected from prostate cancer, breast cancer, and melanoma.

In a twelfth aspect of the present disclosure, provided is a method for inhibiting excessive proliferation of cells, which comprises administering to a subject a therapeutically effective amount of the interfering RNA described above, the delivery system described above, the cell described above, the lipid nanoparticle described above, the medicament or kit described above, or the antibody-nucleic acid drug conjugate described above.

In a thirteenth aspect of the present disclosure, provided is an antimitotic method comprising administering to a subject a therapeutically effective amount of the interfering RNA described above, the delivery system described above, the cell described above, the lipid nanoparticle described above, the medicament or kit described above, or the antibody-nucleic acid drug conjugate described above.

The TUBB2 siRNA sequences screened in the present application can be used for treating diseases related to hyperactive proliferation of cells (e.g., various tumors), demonstrating substantial developmental potential. Experimental validation confirms their ability to significantly inhibit TUBB2 mRNA expression levels in multiple cells (e.g., A375, MDA-MB-231-GFP, PC-3, SKOV-3, and HCT116 cells). Consequently, these sequences are expected to be applicable to diseases related to an increase of tubulin expression, such as various malignant tumors and other disease states. By inhibiting expression of 02-tubulin, they block or reduce proliferation of cells, thereby achieving the objectives of slowing disease progression and improving disease outcomes.

The term “interfering RNA” described in the present disclosure includes a single-stranded RNA (e.g., mature miRNA) or a double-stranded RNA (e.g., siRNA, shRNA, aiRNA, or pre-miRNA), which is capable of reducing or inhibiting the expression of a target gene or sequence (e.g., by mediating degradation and/or inhibiting the translation of an mRNA complementary to the interfering RNA in sequence) when the interfering RNA and the target gene or sequence coexist within the same cell.

The siRNA is a small interfering RNA, and each strand of the molecule comprises a nucleotide of about 15 nt to about 60 nt in length (e.g., nucleotide of about 15-60 nt, 15-50 nt, 15-40 nt, 15-30 nt, 15-25 nt, or 19-25 nt in length, or nucleotide of 15 nt, 16 nt, 17 nt, 18 nt, 19 nt, 20 nt, 21 nt, 22 nt, 23 nt, 24 nt, or 25 nt in length). In one specific embodiment, the siRNA may be chemically synthesized. The siRNA molecule of the present disclosure can silence expression of a target sequence in vitro and/or in vivo. In some embodiments, the siRNA may not contain modified nucleotides; in other embodiments, the siRNA comprises at least one modified nucleotide. For example, the siRNA comprises one, two, three, four, five, six, seven, eight, nine, ten, or more modified nucleotides in the double-strand region. shRNA is a small hairpin RNA or short hairpin RNA comprising short RNA sequences producing tight hairpin turns that can be used to silence gene expression via RNA interference. The shRNA hairpin structure can be lysed by a cellular machinery to an siRNA. An miRNA (microRNA) is a single-strand RNA molecule regulating gene expression with about 21-23 nucleotides in length.

The term “inhibit expression of a target gene” described in the present disclosure refers to the ability of the interfering RNA (e.g., siRNA) of the present disclosure to silence, reduce, or inhibit expression of a target gene (e.g., a TUBB2 gene).

The term “comprise”, “comprising”, or “include” described in the present disclosure is an open-ended description that includes the specified ingredients or steps as described, as well as other specified ingredients or steps that do not substantially affect the technical effect. When the term is used to describe a sequence of a protein or nucleic acid, the protein or nucleic acid may consist of the sequence, or may have additional amino acids or nucleotides at one or two ends of the protein or nucleic acid, while retaining the same or similar activity as the original sequence.

The term “tumor” described in the present disclosure may be any adverse cell proliferation (or any disease that manifests itself as an adverse cell) or neoplasm, or increased predisposition or risk for adverse cell proliferation, neoplasm, or a tumor. The tumor may be benign or malignant, and may also be primary or secondary (metastatic). The neoplasm may be any abnormal growth or proliferation of cells and may be located in any tissue. Examples of the tissue include adrenal gland, adrenal medulla, anus, appendix, bladder, blood, bone, bone marrow, brain, mammary gland, cecum, central nervous system (including or excluding brain), cerebellum, cervix, colon, duodenum, endometrium, gallbladder, esophagus, glial cells, heart, ileum, jejunum, kidney, lacrimal gland, larynx, liver, lung, lymph, lymph nodes, maxilla, mediastinum, mesenterium, myometrium, nasopharynx, omentum, oral cavity, ovary, pancreas, parotid gland, peripheral nervous system, peritoneum, pleura, prostate, salivary glands, colon, skin, soft tissue, spleen, stomach, testis, thymus, thyroid, tongue, tonsils, trachea, uterus, vulva, and rectum. Further preferably, the tumor includes a gastrointestinal tumor (oral cancer, tongue cancer, esophageal cancer, gastric cancer, liver cancer, pancreatic cancer, colorectal cancer, etc.), a nervous system tumor (glioma (e.g., neuroglioma), a neuroepithelial tumor, schwannoma, astrocytoma, neurofibroma (e.g., neurofibrosarcoma), ependymoma, medulloblastoma, meningioma, brain metastasis, etc.), a respiratory tract tumor (nasopharyngeal carcinoma, laryngeal carcinoma, bronchial carcinoma, lung cancer, etc.), a urinary system tumor (e.g., renal cell carcinoma and bladder cancer), a reproductive system tumor (prostate cancer, testis cancer, breast cancer, cervical cancer, ovarian cancer, placental choriocarcinoma, etc.), a hematological and lymphatic system tumor (multiple myeloma, mesothelioma, myeloma, lymphoma (e.g., non-Hodgkin lymphoma, Hodgkin lymphoma, and cutaneous T-cell lymphoma), leukemia (e.g., chronic myelocytic leukemia, acute myeloid leukemia, chronic lymphocytic leukemia, and myelodysplastic syndrome), thymus cancer, etc.), a cutaneous system tumor (e.g., skin cancer, epidermoid cancer, and melanoma), etc., as well as rhabdomyosarcoma, head and neck cancer, etc.

The term “subject” described in the present disclosure may be a human or non-human mammal, and the non-human mammal may be a wild animal, a zoo animal, an economic animal, a companion animal, a laboratory animal, etc. Preferably, the non-human mammal includes, but is not limited to, a pig, a cow, a sheep, a horse, a donkey, a fox, a raccoon dog, a mink, a camel, a dog, a cat, a rabbit, a rodent (e.g., a rat, a mouse, a guinea pig, a hamster, a gerbil, a chinchilla, and a squirrel), a monkey, etc.

The term “treating” described in the present disclosure refers to slowing, interrupting, arresting, controlling, stopping, reducing, or reversing the progression or severity of a sign, symptom, disorder, condition, or disease after the disease has begun to progress, but it does not necessarily involve the complete elimination of all disease-related signs, symptoms, conditions, or disorders.

The term “preventing” described in the present disclosure refers to the practice of preventing or delaying the onset of a disease or condition or symptom in the body.

The term “effective amount” described in the present disclosure refers to an amount or dose of the product of the present disclosure that provides a desired effect (e.g., treating, preventing, or inhibiting TUBB2 expression) after administration to a subject or a cell or an organ thereof in single or multiple doses.

The term “lipid” described in the present disclosure refers to a group of organic compounds that include, but are not limited to, esters of fatty acids and are generally characterized by being poorly soluble in water, but soluble in many organic solvents.

The term “cationic lipid” described in the present disclosure refers to a lipid molecule capable of being positively charged.

The term “neutral lipid” described in the present disclosure refers to an uncharged non-phosphoglyceride lipid molecule.

The term “polyethylene glycol lipid” described in the present disclosure refers to a molecule comprising a lipid portion and a polyethylene glycol portion.

The term “lipid nanoparticle” described in the present disclosure refers to a particle with at least one dimension in the nanometer scale, which comprises at least one lipid.

The term “delivery system” described in the present disclosure refers to a formulation or composition that regulates the spatial, temporal, and dose distribution of a bioactive ingredient in an organism.

### BRIEF DESCRIPTION OF THE DRAWINGS

Hereinafter, examples of the present disclosure will be described in detail with reference to the accompanying drawings, in which:

FIG. 1 shows the mass spectra of the sense strand (left) and the antisense strand (right) of siTUBB2-112.

FIG. 2 shows the mass spectra of the sense strand (left) and the antisense strand (right) of siTUBB2-176.

FIG. 3 shows the mass spectra of the sense strand (left) and the antisense strand (right) of siTUBB2-184.

FIG. 4 shows the mass spectra of the sense strand (left) and the antisense strand (right) of siTUBB2-236.

FIG. 5 shows the mass spectra of the sense strand (left) and the antisense strand (right) of siTUBB2-333.

FIG. 6 shows the mass spectra of the sense strand (left) and the antisense strand (right) of siTUBB2-358.

FIG. 7 shows the mass spectra of the sense strand (left) and the antisense strand (right) of siTUBB2-373.

FIG. 8 shows the mass spectra of the sense strand (left) and the antisense strand (right) of siTUBB2-457.

FIG. 9 shows the mass spectra of the sense strand (left) and the antisense strand (right) of siTUBB2-460.

FIG. 10 shows the mass spectra of the sense strand (left) and the antisense strand (right) of siTUBB2-1092.

FIG. 11 shows the mass spectra of the sense strand (left) and the antisense strand (right) of siTUBB2-1093.

FIG. 12 shows the mass spectra of the sense strand (left) and the antisense strand (right) of siTUBB2-1342.

FIG. 13 shows the mass spectra of the sense strand (left) and the antisense strand (right) of siTUBB2-1345.

FIG. 14 shows the mass spectra of the sense strand (left) and the antisense strand (right) of siTUBB2-598.

FIG. 15 shows the mass spectra of the sense strand (left) and the antisense strand (right) of siTUBB2-982.

FIG. 16 shows the mass spectra of the sense strand (left) and the antisense strand (right) of siTUBB2-984.

FIG. 17 shows the mass spectra of the sense strand (left) and the antisense strand (right) of siTUBB2-985.

FIG. 18 shows the effects of different siRNAs on relative expression of the TUBB2b mRNA in A375 cells.

FIG. 19 shows the effects of different siRNAs on relative expression of the TUBB2 mRNA in A375 cells.

FIG. 20 shows the effects of different siRNAs on relative expression of the TUBB2 mRNA in PC-3 cells.

FIG. 21 shows the results of the cell proliferation inhibition assay of PC-3 cells with different concentrations of the siRNA.

FIG. 22 shows the results of the cell proliferation inhibition assay of A375 cells with different concentrations of the siRNA.

FIG. 23 shows the effect of LNP-siTUBB2 on the relative expression level of the TUBB2 mRNA in MDA-MB-231-GFP cells.

FIG. 24 shows the effects of different concentrations of LNP-siTUBB2 on relative expression of TUBB2 mRNA in PC-3 cells.

FIG. 25 shows the time course of the proliferation inhibition effect of LNP-siTUBB2 on PC-3 cells.

FIG. 26 shows the effect of LNP-siTUBB2 on relative expression of the TUBB2 mRNA in MDA-MB-231-GFP cells.

FIG. 27 shows the cell cycle arrest effect of siTUBB2.

FIG. 28 shows the grouping and administration time and frequency.

FIG. 29 shows the changes in body weight of animals under different administration regimens.

FIG. 30 shows the tumor size under different administration regimens.

FIG. 31 shows the tumor inhibition rates under different administration regimens.

### DETAILED DESCRIPTION

The technical solutions in the examples of the present disclosure will be described clearly and completely below with reference to the drawings. It is obvious that the described examples are only a part of the examples of the present disclosure, rather than all of them. Based on the examples of the present disclosure, all other examples obtained by those of ordinary skill in the art without intensive steps shall fall within the protection scope of the present disclosure.

To detect the inhibitory effects of the siRNA corresponding to the β2-tubulin (TUBB2) mentioned in the present disclosure, the present application sequentially prepared multiple siRNAs targeting TUBB2. In cell experiments, the prepared siRNAs were first delivered into cultured cells, cell samples were harvested at predetermined time points post-transfection, and the RNA was extracted. The extracted RNA was reverse-transcribed into a cDNA, and Ct values were obtained using real-time quantitative PCR. By normalizing data from each group, the relative expression levels of the TUBB2 mRNA were determined, thereby comparing the inhibitory effects of each siRNA on TUBB2 mRNA expression. Additionally, cytotoxicity assays were conducted to validate phenotypic changes in cells, focusing on the mechanistic role of tubulin in cell proliferation and growth. Furthermore, the multiple siRNAs designed above were prepared into lipid nanoparticles and delivered in vivo, demonstrating therapeutic efficacy against tumors.

### Example 1: SiRNA Design and Synthesis

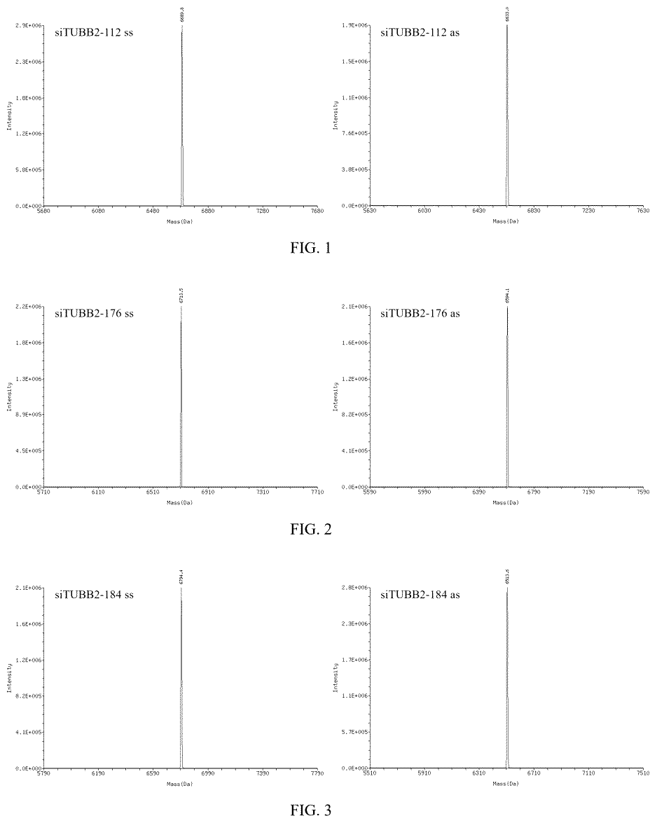

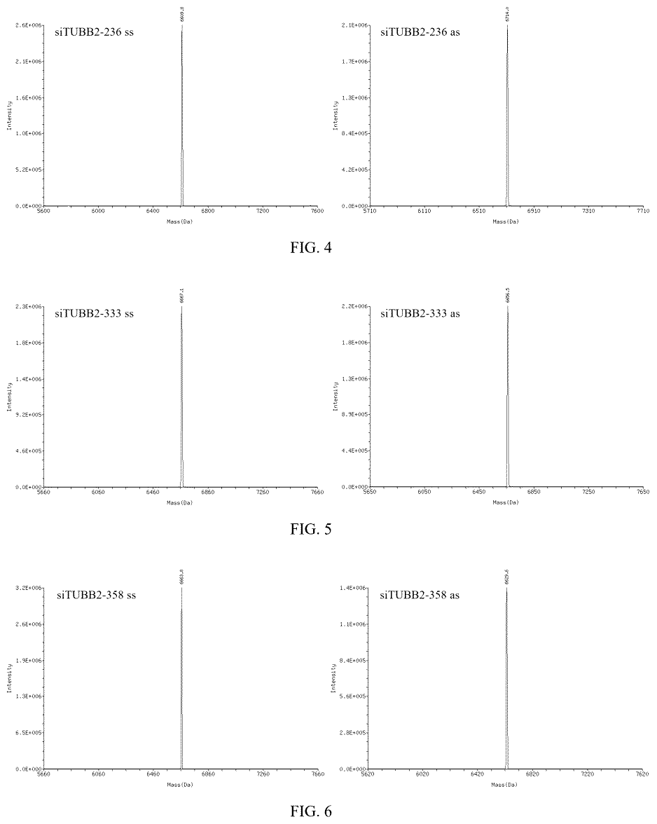

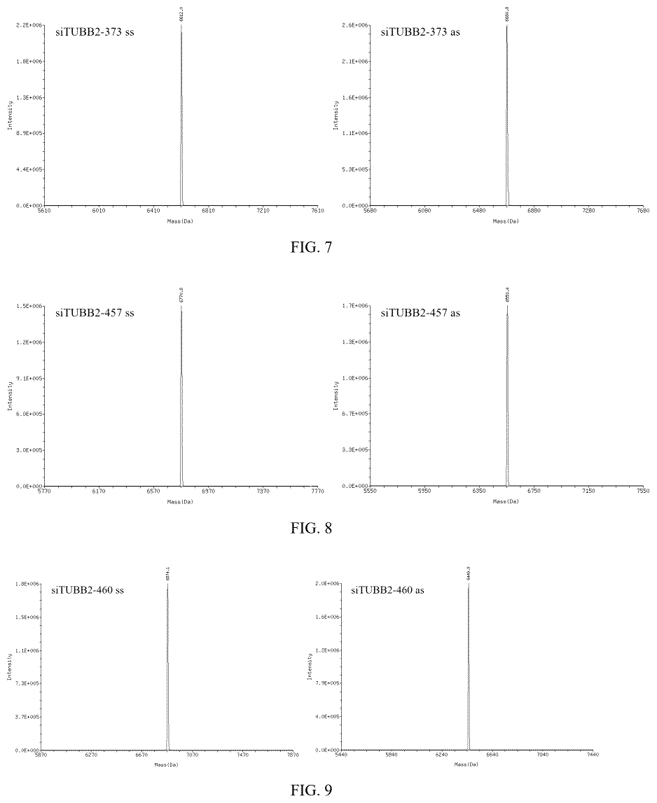

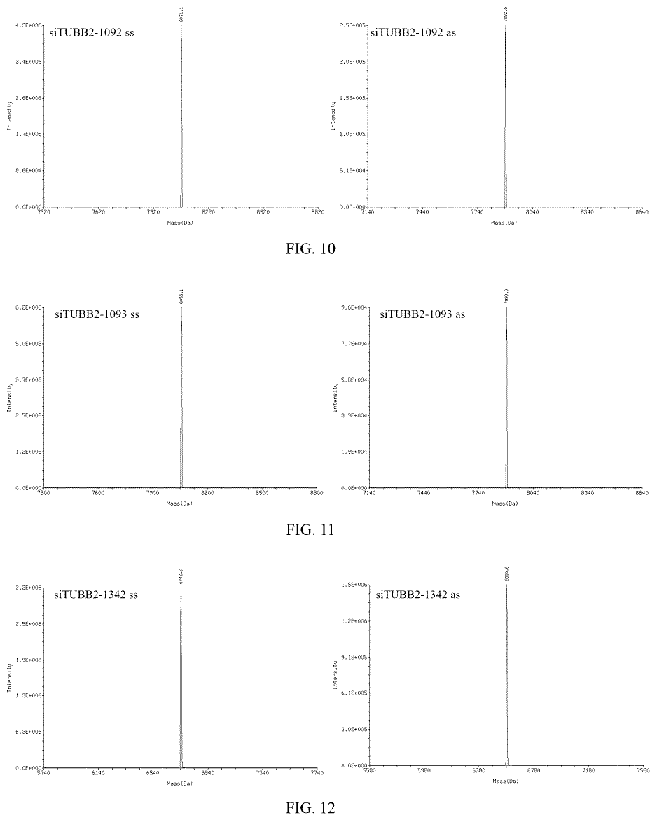

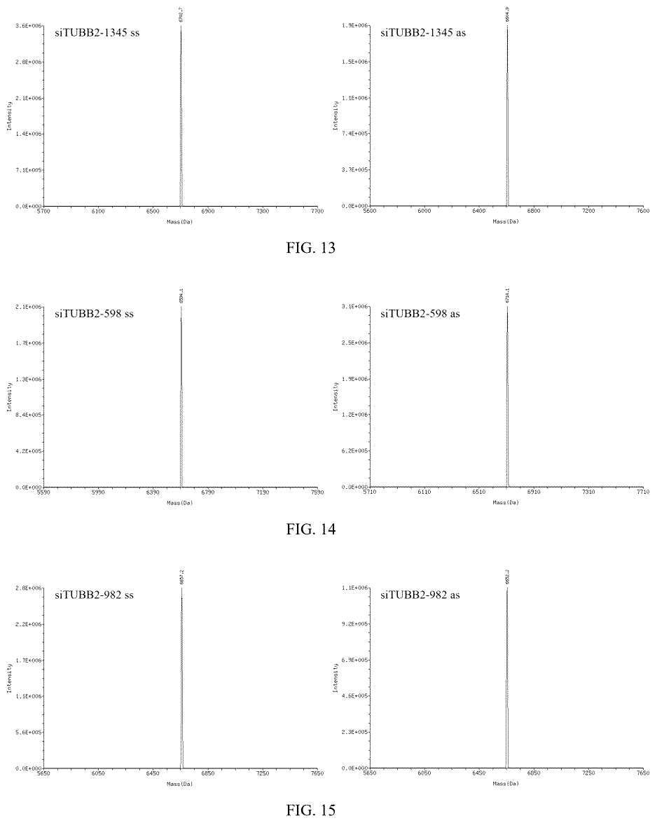

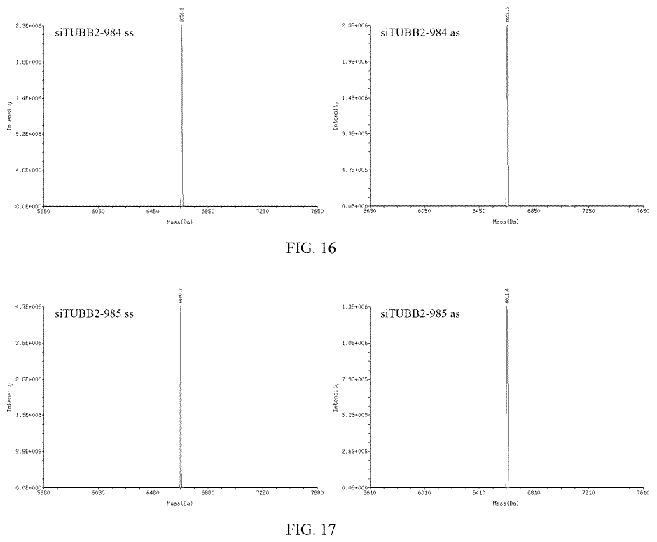

13 siRNA sequences were designed targeting the TUBB2b sequence (GenBank: NM_178012.5), with sequence information shown in Table 1 and mass spectrometry results shown in FIGS. 1-13. For practical use, 2 over-hangs (dTdT) were added to the 3′ ends of the 19-bp siRNA sense and antisense strands, while siRNAs exceeding 19 bp remained unmodified.

TABLE 1
siRNA sequences

| Name | Target site sequence | Sense strand | Antisense strand |
|---|---|---|---|
| siTUBB2- | TGCGTGAGATCGTGCACAT | UGCGUGAGAUCGUGCACA | AUGUGCACGAUCUCACGC |
| 112 | (SEQ ID NO: 1) | U(SEQ ID NO: 14) | A(SEQ ID NO: 27) |
| siTUBB2- | GGTCATCAGTGATGAGCAT | GGUCAUCAGUGAUGAGCA | AUGCUCAUCACUGAUGAC |
| 176 | (SEQ ID NO: 2) | U(SEQ ID NO: 15) | C(SEQ ID NO: 28) |
| siTUBB2- | GTGATGAGCATGGGATTGA | GUGAUGAGCAUGGGAUUG | UCAAUCCCAUGCUCAUCA |
| 184 | (SEQ ID NO: 3) | A(SEQ ID NO: 16) | C(SEQ ID NO: 29) |
| siTUBB2- | GACCATTCGGCCAGATCTT | GACCAUUCGGCCAGAUCU | AAGAUCUGGCCGAAUGGU |
| 236 | (SEQ ID NO: 4) | U(SEQ ID NO: 17) | C(SEQ ID NO: 30) |
| siTUBB2- | TCGGTTAGGTCTGGACCAT | UCGGUUAGGUCUGGACCA | AUGGUCCAGACCUAACCG |
| 333 | (SEQ ID NO: 5) | U(SEQ ID NO: 18) | A(SEQ ID NO: 31) |
| siTUBB2- | AGATCTTCAGACCAGACAA | AGAUCUUCAGACCAGACA | UUGUCUGGUCUGAAGAUC |
| 358 | (SEQ ID NO: 6) | A(SEQ ID NO: 19) | U(SEQ ID NO: 32) |
| siTUBB2- | ACAATTTCGTGTTTGGCCA | ACAAUUUCGUGUUUGGCC | UGGCCAAACACGAAAUUG |
| 373 | (SEQ ID NO: 7) | A(SEQ ID NO: 20) | U(SEQ ID NO: 33) |
| siTUBB2- | TCCTGGATGTGGTGAGGAA | UCCUGGAUGUGGUGAGGA | UUCCUCACCACAUCCAGG |
| 457 | (SEQ ID NO: 8) | A(SEQ ID NO: 21) | A(SEQ ID NO: 34) |
| siTUBB2- | TGGATGTGGTGAGGAAGGA | UGGAUGUGGUGAGGAAGG | UCCUUCCUCACCACAUCCA |
| 460 | (SEQ ID NO: 9) | A(SEQ ID NO: 22) | (SEQ ID NO: 35) |
| siTUBB2- | GAGCAGATGCTCAACGTGC | GAGCAGAUGCUCAACGUG | UGUUCUGCACGUUGAGCA |
| 1092 | AGAACA(SEQ ID NO: 10) | CAGAACA(SEQ ID NO: 23) | UCUGCUC(SEQ ID NO: 36) |
| siTUBB2- | AGCAGATGCTCAACGTGCA | AGCAGAUGCUCAACGUGC | UUGUUCUGCACGUUGAGC |
| 1093 | GAACAA(SEQ ID NO: 11) | AGAACAA(SEQ ID NO: 24) | AUCUGCU(SEQ ID NO: 37) |
| siTUBB2- | CCGAGAGCAACATGAACG | CCGAGAGCAACAUGAACG | UCGUUCAUGUUGCUCUCG |
| 1342 | A(SEQ ID NO: 12) | A(SEQ ID NO: 25) | G(SEQ ID NO: 38) |
| siTUBB2- | AGAGCAACATGAACGACCT | AGAGCAACAUGAACGACC | AGGUCGUUCAUGUUGCUC |
| 1345 | (SEQ ID NO: 13) | U(SEQ ID NO: 26) | U(SEQ ID NO: 39) |

The 13 sequences described above were synthesized by Beijing Tsingke Biotech Co., Ltd. One non-targeting control sequence (siNC, sense strand: 5′-UUC UCC GAA CGU GUC ACG UTT (SEQ ID NO: 40), antisense strand: 5′-ACG UGA AC GUU CGGAGAATT (SEQ ID NO: 41)) was designed and synthesized by Sangon Biotech (Shanghai) Co., Ltd. Additionally, four siRNA sequences resembling those from Patent WO2012177639A3 (sequences 45402, 165110/165111, 165148/165149 (1618322/1618323), corresponding to positions 598, 982, 984, and 985 of the TUBB2b sequence, withmass spectra shown inFIGS. 14-17) were synthesized as controls, shown in Table 2.

TABLE 2
Sequences from Patent WO2012177639A3

| Name | Sense strand | Antisense strand |
|---|---|---|
| siTUBB2- | UCAUGAACACCUUCAGCGU(SEQ ID | ACGCUGAAGGUGUUCAUGA(SEQ ID |
| 598 | NO: 42) | NO: 46) |
| siTUBB2- | AGCAGAUGUUCGACUCCAA(SEQ ID | UUGGAGUCGAACAUCUGCU(SEQ ID |
| 982 | NO: 43) | NO: 47) |
| siTUBB2- | CAGAUGUUCGACUCCAAGA(SEQ ID | UCUUGGAGUCGAACAUCUG(SEQ ID |
| 984 | NO: 44) | NO: 48) |
| siTUBB2- | AGAUGUUCGACUCCAAGAA(SEQ ID | UUCUUGGAGUCGAACAUCU(SEQ ID |
| 985 | NO: 45) | NO: 49) |

### Example 2: Detection of TUBB2 Gene Expression Inhibition

(1) Cell culture and transfection

A375 cells (human melanoma cells) and PC-3 cells (human prostate cancer cells) were used to validate the inhibitory effects of the siRNA. A375 cells were cultured in a DMEM medium containing 10% FBS, 100 U/mL penicillin, and 100 μg/mL streptomycin. PC-3 cells were cultured in an F12K medium containing 10% FBS, 100 U/mL penicillin, and 100 μg/mL streptomycin. Both were cultured in an incubator at 37 °C with 5% CO₂ and saturated humidity. 24 h before the transfection experiment, cells were seeded in a 24-well plate at a density of 8×10⁴ cells/well (A375) or 1.5×10⁵ cells/well (PC-3) and cultured overnight.
(2) 22.5 μL of the OPTI-MEM medium was taken to dilute 2.5 μL of the siTUBB2 or the siNC (stock solution concentration: 10 μM). 23.5 μL of the OPTI-MEM medium was taken to dilute 1.5 μL of the Lipofectamine RNAiMAX transfection reagent. The two diluted solutions were combined, gently mixed, and allowed to stand for 5 min. Additionally, a blank group and an NC group were set.
(3) The medium for the cells in each well of the cell plate was replaced with an antibiotic-free medium, with a volume of 450 μL per well. Subsequently, 50 μL of the mixed solution described above was added to each well, bringing the final volume in each well to 500 μL. The transfection concentration of the siTUBB2 (or siNC) was 50 nM in all cases.
(4) 48 h after transfection, the 24-well plate was taken out from the incubator at 37 °C with 5% CO₂, and the cells were harvested for RNA extraction for subsequent detection.
(5) RNA extraction: The RNA was extracted using the RNA extraction kit from Promega. Briefly, after the cells were washed with PBS, 300 μL of a lysis solution was added, and the cells were pipetted using a pipettor. After complete lysis, 300 μL of a diluent was added, and the mixture was incubated in a water bath at 70 °C for 3 min. The mixture was centrifuged at 14,000 rpm for 10 min. The supernatant was transferred into a new 1.5-mL EP tube, and 300 μL of absolute ethanol was added. The mixture was uniformly mixed and added into an adsorption column. The mixture was centrifuged at 14,000 rpm for 1 min. The filtrate was discarded, and 600 μL of a washing solution was added. The mixture was centrifuged for another 1 min. The filtrate was discarded, 50 μL of a freshly prepared DNase reaction solution was added to each well, and the mixture was left to stand at room temperature for 15 min. Subsequently, 600 μL of a washing solution was added, and the mixture was centrifuged for 1 min. The filtrate was discarded, 600 μL of a washing solution was added, and the mixture was centrifuged for another 1 min. After that, the filtrate was discarded, the residue was centrifuged for 2 min, and 50 μL of nuclease-free water was added to each well. The mixture was left to stand at room temperature for 5 min and subsequently centrifuged for 2 min to harvest eluted RNA.

Quality control of RNA: The RNA content and purity were detected by Nanodrop, and RNA integrity was detected by 1% agarose gel electrophoresis.
(6) RNA reverse transcription

Total RNA extracted from the sample was used as a template; using a LABLEAD reverse transcription kit, an RNA reverse transcription system shown in Table 3 was established.

TABLE 3
RNA reverse transcription system

| Reagent | Amount of use |
|---|---|
| All-in-one First Strand Synthesis MasterMix | 4 μL |
| dsDNase | 1 μL |
| RNA template | 100 ng~1 μg |
| RNase-free H₂O | Make up to 20 μL |

The above system was thoroughly mixed and centrifuged to allow the liquid to be all at the bottom of the tube. The reaction was performed at 37 °C for 2 min, 55 °C for 15 min, and 85 °C for 5 min. The resulting product was the cDNA template and was stored on ice for subsequent use.
(7) Quantitative fluorescence PCR detection

Using the TB green Premix Ex Taq II (Tli RNaseH Plus) (Takara) reagent or other equivalent reagents, the reaction system shown in Table 4 was established.

TABLE 4
Quantitative fluorescence PCR reaction system

| Reagent | Amount of addition |
|---|---|
| 2 × SYBR Green Mix | 10 μL |
| Forward primer (10 μM) | 0.5 μL |
| Reverse primer (10 μM) | 0.5 μL |
| cDNA | 50~100 ng |
| RNase-free H₂O | Make up volume to 20 μL |

PCR amplification was performed according to the following procedure, and the primer sequences are shown in Table 5.
Pre-denaturation at 95 °C for 10 min, followed by the cycle as follows:
*95 °C 10 s
60 °C 20 s
70 °C 10 s
Plate reading
Return to * for 40 cycles in total.
Plot a melting curve: read the plate every 0.5 °C and then stop for 5 s between 65 °C and 95 °C.

TABLE 5
Primer sequences

| | | | PCR |
|---|---|---|---|
| | | | amplification |
| Gene | Primer | Sequence | length (bp) |
| TUBB2 | TUBB2-1F | GCCAGATCTTCAGACCAGAC(SEQ ID NO: 50) | 125 |
| | TUBB2-1R | ACTCCTTCCTCACCACATCC(SEQ ID NO: 51) | |
| | TUBB2-2F | GGCACGATGGATTCGGTTAGG(SEQ ID NO: 52) | 63 |
| | TUBB2-2R | ACACGAAATTGTCTGGTCTGAAG(SEQ ID NO: 53) | |
| GAPDH | GAPDHF | CATGAGAAGTATGACAACAGCCT(SEQ ID NO: 54) | 113 |
| | GAPDHR | AGTCCTTCCACGATACCAAAGT(SEQ ID NO: 55) | |

Note:
The primer sequences TUBB2-1F and TUBB2-1R can amplify templates of both TUBB2a and TUBB2b, whereas the primer sequences TUBB2-2F and TUBB2-2R can only amplify the TUBB2b template.
8. Inhibitory effect

The relative expression level of the TUBB2 mRNA or the TUBB2b mRNA was calculated by ΔΔCt with GAPDH as an endogenous reference gene; mRNA expression levels were normalized for each group with the expression level of the blank group as 100%.

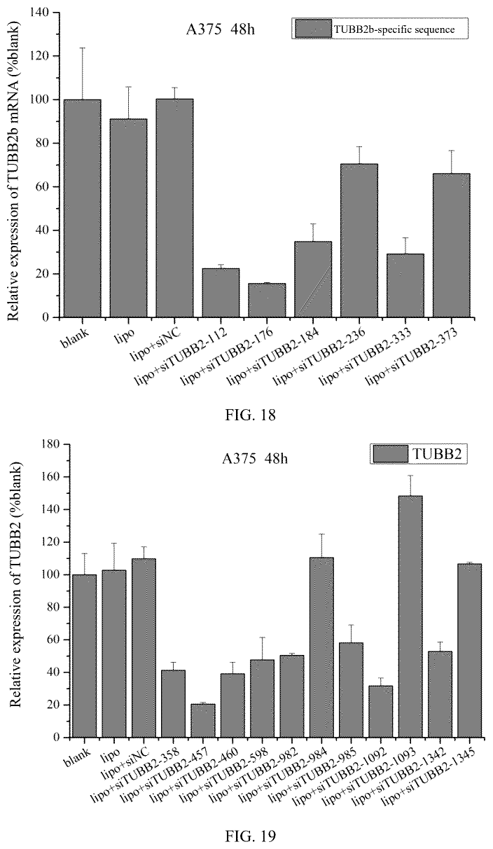

The relative expression levels of the mRNA of each group of cells are shown in Tables 6, 7, and 8 below and FIGS. 18, 19, and 20 below.

The results demonstrated that in A375 cells, the relative expression of TUBB2b or TUBB2 was inhibited in cells treated with the siRNAs designed in Table 1 of Example 1, except for siTUBB2-1093 and siTUBB2-1345. Furthermore, all siRNA sequences except siTUBB2-236, siTUBB2-373, and siTUBB2-1342 exhibited superior inhibitory effects compared with the control check sequences. Notably, the relative expression levels of TUBB2b (siTUBB2-112, siTUBB2-184, siTUBB2-176, and siTUBB2-333) or TUBB2 (siTUBB2-457 and siTUBB2-1092) mRNA were all significantly inhibited compared with the blank group and siNC group.

TABLE 6
Amplification results using TUBB2F and TUBB2R primers

| A375 (TUBB2b-specific sequences) | mean | SD | CV |
|---|---|---|---|
| blank | 100.00 | 23.76 | 23.76 |
| Lipofectamine RNAiMAX | 91.11 | 14.77 | 16.21 |
| Lipofectamine RNAiMAX + siNC | 100.34 | 5.21 | 5.19 |
| Lipofectamine RNAiMAX + siTUBB2-112 | 22.48 | 1.76 | 7.82 |
| Lipofectamine RNAiMAX + siTUBB2-176 | 15.61 | 0.47 | 3.01 |
| Lipofectamine RNAiMAX + siTUBB2-184 | 34.79 | 8.14 | 23.39 |
| Lipofectamine RNAiMAX + siTUBB2-236 | 70.45 | 7.97 | 11.31 |
| Lipofectamine RNAiMAX + siTUBB2-333 | 29.14 | 7.45 | 25.56 |
| Lipofectamine RNAiMAX + siTUBB2-373 | 66.04 | 10.63 | 16.10 |

TABLE 7
Amplification results using TUBB1F and TUBB1R primers

| A375 (TUBB2a and 2b | | | |
|---|---|---|---|
| consensus sequence) | mean | SD | CV |
| blank | 100.00 | 12.99 | 12.99 |
| Lipofectamine RNAiMAX | 102.65 | 16.62 | 16.19 |
| Lipofectamine RNAiMAX + siNC | 109.75 | 7.41 | 6.75 |
| Lipofectamine RNAiMAX + siTUBB2-358 | 41.24 | 5.11 | 12.39 |
| Lipofectamine RNAiMAX + siTUBB2-457 | 20.48 | 1.07 | 5.24 |
| Lipofectamine RNAiMAX + siTUBB2-460 | 39.14 | 7.10 | 18.14 |
| Lipofectamine RNAiMAX + siTUBB2-598 | 47.71 | 13.72 | 28.75 |
| Lipofectamine RNAiMAX + siTUBB2-982 | 50.36 | 1.36 | 2.71 |
| Lipofectamine RNAiMAX + siTUBB2-984 | 110.46 | 14.59 | 13.21 |
| Lipofectamine RNAiMAX + siTUBB2-985 | 58.05 | 11.19 | 19.27 |
| Lipofectamine RNAiMAX + siTUBB2-1092 | 31.69 | 4.87 | 15.38 |
| Lipofectamine RNAiMAX + siTUBB2-1093 | 148.25 | 12.56 | 8.47 |
| Lipofectamine RNAiMAX + siTUBB2-1342 | 52.83 | 5.80 | 10.99 |
| Lipofectamine RNAiMAX + siTUBB2-1345 | 106.59 | 0.92 | 0.86 |

In PC-3 cells, the relative expression of TUBB2b or TUBB2 was inhibited in cells treated with the siRNAs designed in Table 1 of Example 1. Illustratively, siTUBB2-176, siTUBB2-457, and siTUBB2-1092 significantly inhibited TUBB2 mRNA expression in cells, with siTUBB2-457 and siTUBB2-1092 inhibiting about 90% of TUBB2 mRNA expression.

TABLE 8
Inhibitory effects of some sequences on
TUBB2 mRNA expression in PC-3 cells

| | Relative expression levels | |
|---|---|---|
| | of TUBB2 mRNA | |
| PC-3 | mean | SD |
| blank | 100 | 7.37 |
| lipofectamineRNAiMAX | 98.31 | 1.64 |
| lipofectamineRNAiMAX + siNC | 129.35 | 30.07 |
| lipofectamineRNAiMAX + siTUBB2-176 | 32.74 | 4.57 |
| lipofectamineRNAiMAX + siTUBB2-457 | 8.59 | 0.95 |
| lipofectamineRNAiMAX − siTUBB2-1092 | 11.09 | 2.93 |

### Example 3: Cell Proliferation Inhibition Assay

(1) A375 cells were cultured in a DMEM medium containing 7.5% FBS, 100 U/mL penicillin, and 100 μg/mL streptomycin. PC-3 cells were cultured in an F12K medium containing 10% FBS, 100 U/mL penicillin, and 100 μg/mL streptomycin. Both were cultured in an incubator at 37 °C with 5% CO₂ and saturated humidity. The cells were cultured in an incubator at 37 °C with 5% CO₂ and saturated humidity. 24 h before the experiment, cells were seeded in a 96-well plate at a density of 2.5×10³ cells/well (A375) or 1×10⁴ cells/well (PC-3) and cultured overnight.
(2) 4.5 μL of the OPTI-MEM medium was taken to dilute 0.5 μL of the siTUBB2 or the siNC (stock solution concentration: 10 μM). 4.7 μL of the OPTI-MEM medium was taken to dilute 0.3 μL of the Lipofectamine RNAiMAX transfection reagent. The two diluted solutions were combined, gently mixed, and allowed to stand for 5 min. For testing the cell proliferation inhibitory effects of siTUBB2 at different concentrations, corresponding amounts of use were calculated based on the final concentration. In addition, a blank group, a lipofectamine RNAiMAX group, and a lipofectamine RNAiMAX+NC group were set.
(3) The medium for the cells in each well of the cell plate was replaced with an antibiotic-free medium, with a volume of 90 μL per well. Subsequently, 10 μL of the mixed solution described above was added to each well, bringing the final volume in each well to 100 μL. The transfection concentration of the siTUBB2 (or siNC) was 50 nM in all cases.
(4) 48 h or 72 h after transfection, the 96-well plate was taken out from the incubator at 37 °C with 5% CO₂, and 10 μL of CCK-8 was added to each well. The plate was further cultured in the incubator at 37 °C with 5% CO₂ for about 4 h, and absorbance at 450 nm was detected using a microplate reader.

Cell viability %=100×Aₑₓₚₑᵣᵢₘₑₙₜₐₗ/A_{control}

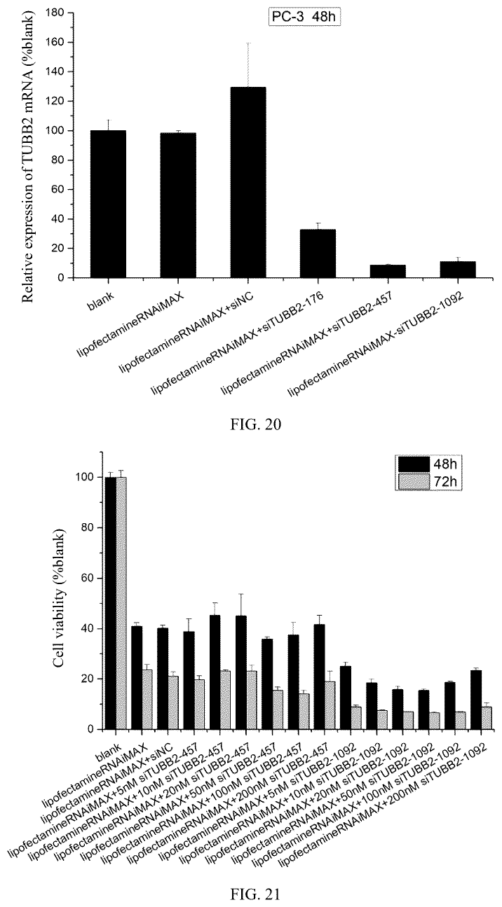

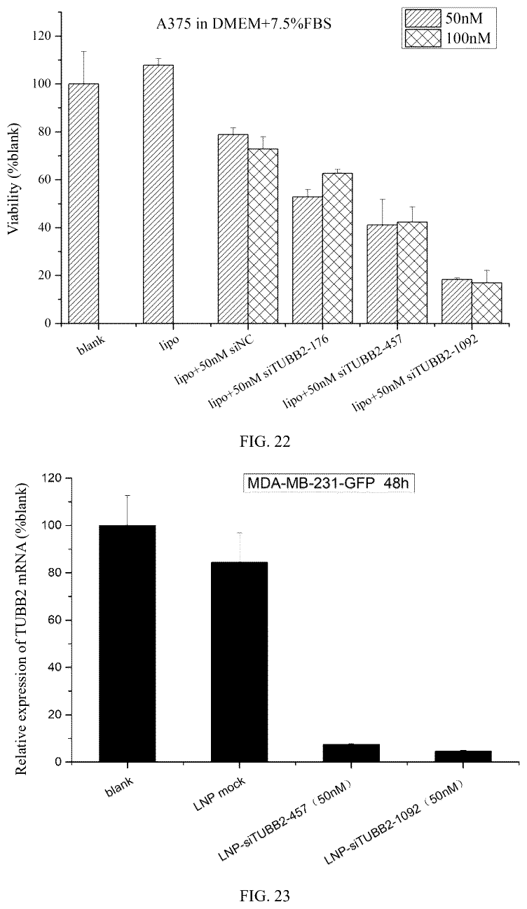

The results demonstrated that all siTUBB2 designed in Table 1 of Example 1 could kill tumor cells. Illustratively, in cells treated with siTUBB2-1092, cell viability was only 16%-18%; in cells treated with siTUBB2-457, cell viability was only about 40%; in the siTUBB2-176 treatment group, cell viability was only 50%-60%. Therefore, it can be concluded that siTUBB2-176, siTUBB2-457, and siTUBB2-1092 can significantly inhibit cell proliferation (see Tables 9 and 10, FIGS. 22 and 21).

TABLE 9
A375 cell proliferation inhibition assay

| | Viability % | |
|---|---|---|
| A375(DMEM + 7.5% FBS) | mean | SD |
| blank | 100.000 | 13.540 |
| Lipofectamine RNAiMAX | 107.818 | 2.874 |
| Lipofectamine RNAiMAX + 50 nM siNC | 78.834 | 2.841 |
| Lipofectamine RNAiMAX + 100 nM siNC | 72.809 | 5.200 |
| Lipofectamine RNAiMAX + 50 nM siTUBB2-176 | 52.865 | 3.101 |
| Lipofectamine RNAiMAX + 100 nM siTUBB2-176 | 62.627 | 1.664 |
| Lipofectamine RNAiMAX + 50 nM siTUBB2-457 | 41.155 | 10.698 |
| Lipofectamine RNAiMAX + 100 nM siTUBB2-457 | 42.344 | 6.361 |
| Lipofectamine RNAiMAX + 50 nM siTUBB2-1092 | 18.331 | 0.663 |
| Lipofectamine RNAiMAX + 100 nM siTUBB2-1092 | 16.887 | 5.306 |

TABLE 10
PC-3 cell proliferation inhibition assay

| | 48 h | | 72 h | |
|---|---|---|---|---|
| PC-3 | mean | SD | mean | SD |
| blank | 100 | 2.08 | 100 | 2.7 |
| lipo | 40.95 | 1.4 | 23.73 | 2.09 |
| lipo + siNC | 40.17 | 1.25 | 21.06 | 1.99 |
| Lipofectamine RNAiMAX + 5 nM siTUBB2-457 | 38.95 | 5.1 | 19.75 | 1.57 |
| Lipofectamine RNAiMAX + 10 nM siTUBB2-457 | 45.32 | 4.89 | 23.17 | 0.42 |
| Lipofectamine RNAiMAX + 20 nM siTUBB2-457 | 45.1 | 8.63 | 23.25 | 2.4 |
| Lipofectamine RNAiMAX + 50 nM siTUBB2-457 | 35.93 | 0.79 | 15.47 | 1.47 |
| Lipofectamine RNAiMAX + 100 nM siTUBB2-457 | 37.53 | 4.93 | 14.13 | 1.46 |
| Lipofectamine RNAiMAX + 200 nM siTUBB2-457 | 41.58 | 3.82 | 19.09 | 4.02 |
| Lipofectamine RNAiMAX + 5 nM siTUBB2-1092 | 25.1 | 1.49 | 8.94 | 0.73 |
| Lipofectamine RNAiMAX + 10 nM siTUBB2-1092 | 18.48 | 1.5 | 7.55 | 0.17 |
| Lipofectamine RNAiMAX + 20 nM siTUBB2-1092 | 15.86 | 1.38 | 7.11 | 0.02 |
| Lipofectamine RNAiMAX + 50 nM siTUBB2-1092 | 15.53 | 0.53 | 6.71 | 0.29 |
| Lipofectamine RNAiMAX + 100 nM siTUBB2-1092 | 18.7 | 0.42 | 6.96 | 0.08 |
| Lipofectamine RNAiMAX + 200 nM siTUBB2-1092 | 23.52 | 0.74 | 80.95 | 1.69 |

### Example 4: LNP Preparation, Biological Activity, and Cell Proliferation Inhibitory Activity

### Evaluation

(1) Preparation of LNP-siTUBB2

71.02 mg of SM-102 was weighed out and dissolved in 10 mL of EtOH to give an SM-102 stock solution.

249.5 mg of M-DMG-2000 was weighed out and dissolved in 10 mL of anhydrous EtOH to give a PEG component stock solution.

79.02 mg of DSPC was weighed out and dissolved in 10 mL of anhydrous EtOH to give a DSPC stock solution.

38.66 mg of cholesterol was weighed out and dissolved in 10 mL of anhydrous EtOH to give a cholesterol stock solution.

2160 μL of the SM-102 stock solution, 64.8 μL of the PEG component stock solution, 432 μL of the DSPC stock solution, and 1664 μL of the cholesterol stock solution were taken and well mixed to give an LNP/EtOH solution.

150 nmol of siTUBB2 in Table 1 of Example 1 was taken, and 6.5 mL of a 50 mM citric acid buffer (pH=4.0) was added to give an siRNA/buffer solution.

Microfluidic preparation: The LNP/EtOH solution was used in channel 1, with a solution volume of 2 mL and a flow rate of 5 mL/min; the siRNA/buffer solution was used in channel 2, with a solution volume of 6 mL and a flow rate of 15 mL/min.

4 mL of the microfluidic preparation solution was taken and dialyzed with PBS (pH=7.4) for 16 h to give LNP-siTUBB2, resulting in a final siTUBB2 concentration of 0.2 mg/mL.
(2) Particle size and zeta potential detection of LNP-siTUBB2 using a nanoparticle size analyzer The particle size and zeta potential of LNP-siTUBB2 were detected using a NanoBrook nanoparticle size analyzer (Brookhaven Instruments Corporation, USA). 100 μL of the prepared LNP-siTUBB2 was diluted to 2 mL with DEPC water, and the mixture was loaded for measurement. Each sample was tested three times, and the mean value of effective particle size (D50) was used to represent the particle size. Illustrative results are shown in Table 11.

TABLE 11
Effective particle size and zeta potential
of LNP-siTUBB2-457 and LNP-siTUBBb-1092

| | Effective particle | | Zeta potential |
|---|---|---|---|
| | size (nm) | Polydispersity | (mV) |
| LNP-TUBB2-457 | 104.53 ± 0.62 | 0.25 ± 0.00 | 1.31 ± 0.00 |
| LNP-TUBB2-1092 | 92.86 ± 0.60 | 0.12 ± 0.03 | 9.93 ± 1.21 |

The results demonstrated that the effective particle sizes of both LNPs were about 100 nm, and their zeta potentials were positively charged.
(3) Biological activity evaluation of LNP-siTUBB2

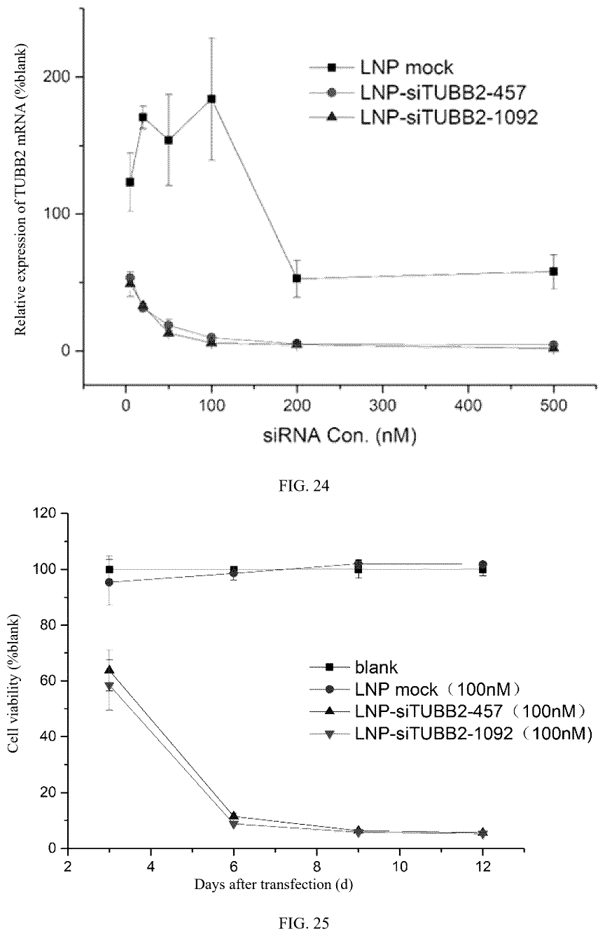

The method was the same as that in Example 2, using MDA-MB-231-GFP human breast cancer cells (cultured in a 1640 medium containing 10% FBS and 1% penicillin-streptomycin) or PC-3 cells (cultured in an F12K medium containing 10% FBS and 1% penicillin-streptomycin). The results demonstrated that LNPs could efficiently deliver siTUBB2 into MDA-MB-231-GFP or PC-3 cells and significantly inhibit TUBB2 mRNA expression (see Tables 12 and 13, and FIGS. 23 and 24).

TABLE 12
Effect of LNP-siTUBB2 on TUBB2 mRNA
expression in MDA-MB-231-GFP cells

| | Relative expression of TUBB2 mRNA | |
|---|---|---|
| MDA-MB-231-GFP | mean | SD |
| blank | 100 | 12.53 |
| LNP mock | 84.45 | 12.36 |
| LNP-siTUBB2-457(50 nM) | 7.49 | 0.18 |
| LNP-siTUBB2-1092(50 nM) | 4.54 | 0.42 |

TABLE 13
Effect of LNP-siTUBB2 on TUBB2
mRNA expression in PC-3 cells

| | Relative expression of TUBB2 mRNA | |
|---|---|---|
| PC-3 | mean | SD |
| blank | 100 | 11.08 |
| LNP mock(5 nm) | 123.38 | 21.24 |
| LNP mock(20 nm) | 170.6 | 8.32 |
| LNP mock(50 nm) | 154.23 | 33.23 |
| LNP mock(100 nm) | 184.03 | 44.96 |
| LNP mock(200 nm) | 52.7 | 13.56 |
| LNP mock(500 nm) | 57.98 | 12.58 |
| LNP-siTUBB2-457(5 nm) | 53.25 | 1.39 |
| LNP-siTUBB2-457(20 nm) | 31.27 | 2.04 |
| LNP-siTUBB2-457(50 nm) | 18.7 | 4.41 |
| LNP-siTUBB2-457(100 nm) | 9.75 | 1.84 |
| LNP-siTUBB2-457(200 nm) | 5.44 | 1.38 |
| LNP-siTUBB2-457(500 nm) | 4.39 | 0.33 |
| LNP-siTUBB2-1092(5 nm) | 48.88 | 9.2 |
| LNP-siTUBB2-1092(20 nm) | 32.87 | 2 |
| LNP-siTUBB2-1092(50 nm) | 12.92 | 1.98 |
| LNP-siTUBB2-1092(100 nm) | 5.87 | 1.42 |
| LNP-siTUBB2-1092(200 nm) | 4.61 | 2.21 |
| LNP-siTUBB2-1092(500 nm) | 1.67 | 0.45 |

(4) Cell proliferation inhibitory activity evaluation of LNP-siTUBB2

The method was the same as that in Example 3, using PC-3 cells for testing. The results demonstrated that LNP-siTUBB2 could significantly inhibit the proliferation of PC-3 cells, with cell viability being significantly reduced and the effect sustained over an extended period see Table 14 and FIG. 25).

TABLE 14
Proliferation inhibitory effects of LNP-siTUBB2 on PC-3 cells

| | 3 d | | 6 d | | 9 d | | 12 d | |
|---|---|---|---|---|---|---|---|---|
| PC-3 | mean | SD | mean | SD | mean | SD | mean | SD |
| Blank | 100 | 4.98 | 100 | 0.47 | 100 | 3.18 | 100 | 2.36 |
| LNP-mock(100 nM) | 95.37 | 8.11 | 98.59 | 2.55 | 102.02 | 1.62 | 101.77 | 0.86 |
| LNP-siTUBB2-457(100 nM) | 63.7 | 7.38 | 11.5 | 1.06 | 6.37 | 0.3 | 5.69 | 0.2 |
| LNP-siTUBB2-1092(100 nM) | 58.47 | 9.05 | 8.81 | 0.15 | 5.84 | 0.1 | 5.3 | 0.12 |

### Example 5: Chemical Modifications of siTUBB2, Biological Effect Evaluation, and Cell Proliferation Inhibition Evaluation

siTUBB2-457 and siTUBB2-1092 were selected from Table 1 of Example 1 for chemical modifications, including methylation, fluorination, a phosphorothioate modification, a pseudouridine modification, etc. (see Table 15). The designed siTUBB2 with specified modifications sequences were synthesized by Sangon Biotech (Shanghai) Co., Ltd.

TABLE 15
Modifications of siTUBB2 modified sequences

| Gene | | |
|---|---|---|
| name | Sense strand (5′-3′) | Antisense strand (5′-3′) |
| TUBB2- | 5′- | 5′- |
| 457-1 | (mU)(mC)(mC)(mU)GGA(mU)G(mU) | (mU)(mU)(mC)(mC)(mU)(mC)A(mC) |
| | GG(mU)GA GGA AdTdT-3′(SEQ ID | (mC)A(mC)A(mU)(mC)(mC) AGG |
| | NO: 56) | AdTdT-3′(SEQ ID NO: 60) |
| TUBB2- | 5′- | 5′- |
| 457-4 | ΨCCΨGGAΨGΨGGΨGAGGAAdTdT-3′ | ΨΨCCΨCACCACAΨCCAGGAdTdT- |
| | (SEQ ID NO: 57) | 3′(SEQ ID NO: 61) |
| TUBB2- | 5′- | 5′-ΨGΨΨCΨGCA CGΨΨGA |
| 1092-4 | GAGCAGAΨGCΨCAACGΨGCAGA | GCAΨCΨGCΨC-3′(SEQ ID NO: 62) |
| | ACA-3′(SEQ ID NO: 58) | |
| TUBB2- | 5′-(mG)-(mA)- | 5′-(fU)-(fG)- |
| 1092-6 | (mG)(fC)(mA)(fG)(mA)(fU)(mG)(fC) | (fU)(mU)(fC)(mU)(fG)(mC)(fA)(mC) |
| | (fU)(fC)(mA)(fA)(mC)(fG)(mU)(fG) | (mG)(mU)(fU)(mG)(fA)(mG)(fC)(mA)(fU) |
| | (mC)(fA)(mG)(fA)(mA)(mC)(mA)- | (mC)(fU)(mG)(fC)-(fU)-(fC)-3′(SEQ ID |
| | 3′(SEQ ID NO: 59) | NO: 63) |

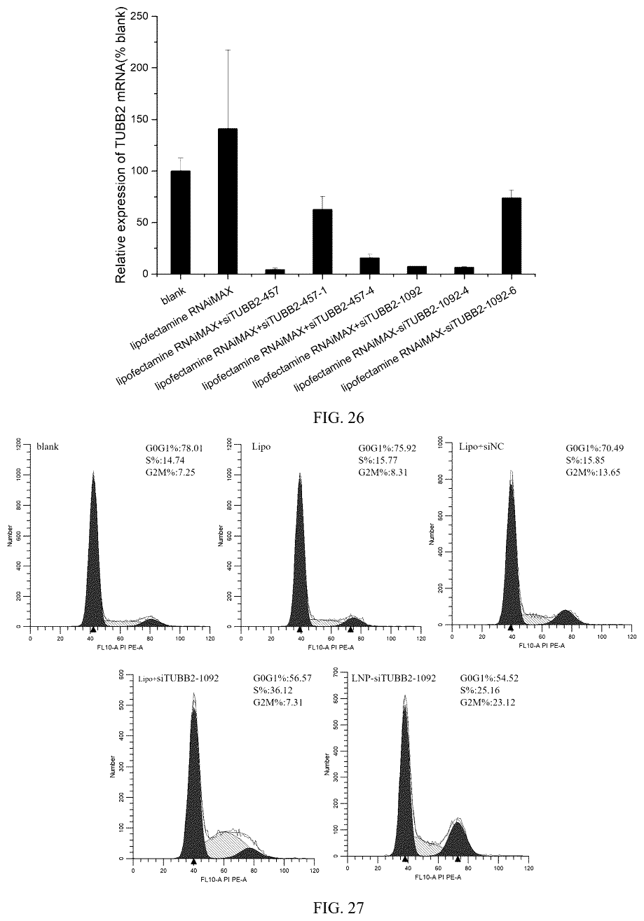

m is a methyl modification (2′-O-methyl); f is a fluorination modification (2′-deoxy-2′-fluoro); is a phosphorothioate modification (p); Ψ is a pseudouridine modification (pseudouridine). The experimental method for evaluating the biological effects of modified siTUBB2 was the same as that in Example 2. Among the modified siTUBB2 sequences, siTUBB2-457-4 and siTUBB2-1092-4 exhibited biological effects comparable to those of unmodified siTUBB2 (Table 16 and FIG. 26).

TABLE 16
Effects of modified siTUBB2 sequences on relative
expression of TUBB2 mRNA in MDA-MB-231-GFP

| | Relative expression of | |
|---|---|---|
| | TUBB2 mRNA | |
| MDA-MB-231-GFP | mean | SD |
| blank | 100 | 12.53 |
| lipofectamine RNAiMAX | 140.84 | 76.54 |
| lipofectamine RNAiMAX + siTUBB2-457 | 4.38 | 1.3 |
| lipofectamine RNAiMAX + siTUBB2-457-1 | 62.65 | 12.75 |
| lipofectamine RNAiMAX + siTUBB2-457-4 | 15.65 | 3.85 |
| lipofectamine RNAiMAX + siTUBB2-1092 | 7.49 | 0.18 |
| lipofectamine RNAiMAX − siTUBB2-1092-4 | 6.6 | 0.38 |
| lipofectamine RNAiMAX − siTUBB2-1092-6 | 73.87 | 7.81 |

### Example 6: Cell Cycle Arrest Assay

72 h after transfecting A375 cells with siTUBB2-1092 using the method described in Example 2, cells were washed with PBS and digested with pancreatin to detach the cells, and the digestion was terminated with a serum-containing medium. The mixture was centrifuged at 300×g, and the supernatant was discarded. The cells were washed twice with PBS, and the supernatant was discarded. The cells were resuspended in 70% ice-cold ethanol (pre-cooled at 4 °C), gently pipetted, mixed well, sealed with parafilm, and stored overnight at 4 °C. The next day, the cells were centrifuged at 500×g for 5 min, and the supernatant was removed. The cells were washed twice with 2 mL of PBS and centrifuged at 500×g for 5 min, and the supernatant was removed. 100 μL of 100 μg/mL RNase A and 400 μL of a 50 μg/mL PI solution were added to each tube, followed by incubation at 37 °C in the dark for 30 min. 2 mL of PBS was added to wash the cells. The cells were centrifuged at 500×g for 5 min, and the supernatant was discarded. 0.5 mL of PBS was added to resuspend the cells. Flow cytometry analysis was performed with 10⁶ counted cells. Red fluorescence was detected at an excitation wavelength of 488 nm, and cell cycle phase distribution was analyzed using the FlowJo software.

The results demonstrated that siTUBB2-1092 could induce significant cell cycle arrest in A375 cells with a decrease in the proportion of cells in the G0G1% phase and an arrest in the G2M phase (FIG. 27).

### Example 7: Tumor Inhibition Assay

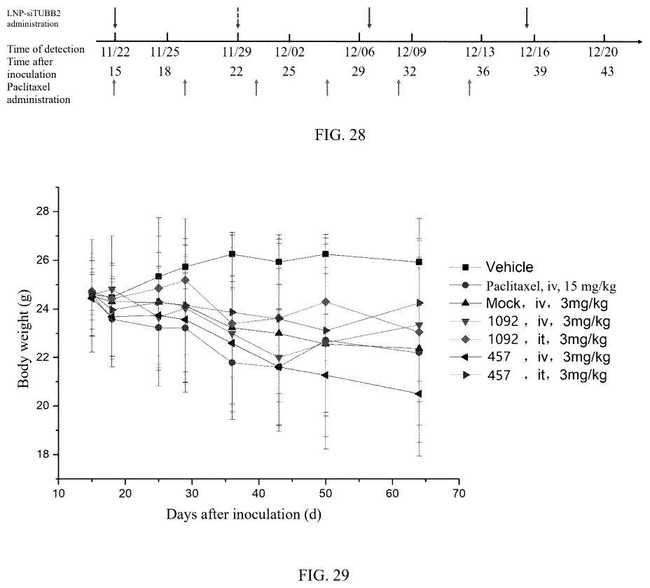

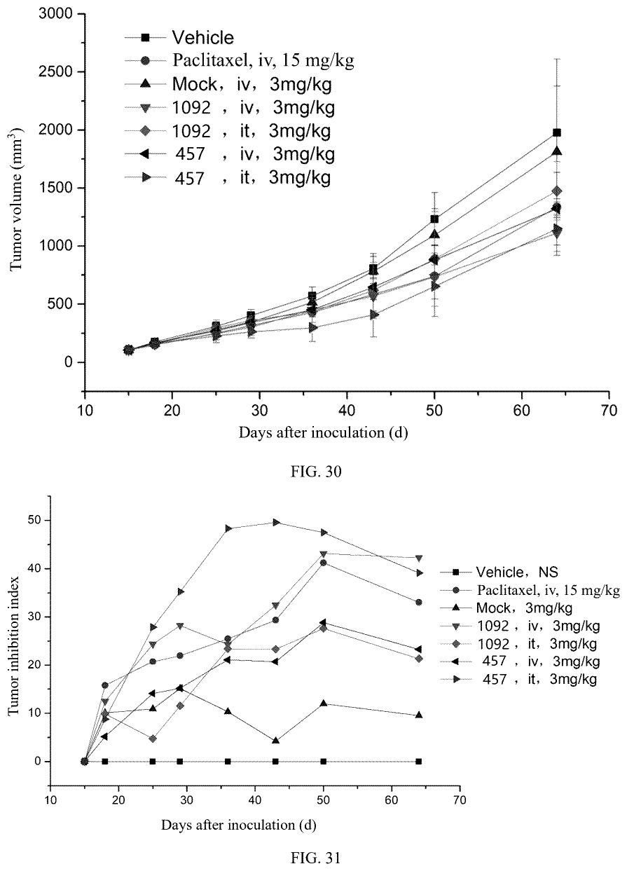

The human prostate cancer cell line PC-3 was cultured in an F-12K culture liquid containing 10% fetal bovine serum in a cell incubator at 37 °C with 5% CO₂. The cells were subcultured by passaging into new flasks every 3-4 days after reaching full confluence, and tumor cells in the logarithmic growth phase were used for in vivo tumor inoculation. PC-3 tumor cells were resuspended at a concentration of 5×10⁷ cells/mL and inoculated subcutaneously on the right flank of Balb/c nude mice (100 μL/mouse). When tumors reached an average volume of about 100-150 mm³, animals were divided into 7 groups (6 mice/group) for administration. Grouping and administration dose and frequency are detailed in Table 17 and FIG. 28. Body weight was weighed periodically (FIG. 29), tumor size was detected (FIG. 30), and tumor inhibition rates (TGI) were calculated (FIG. 31).

TABLE 17
Animal grouping and administration regimens

| | | Number of | Dose | Route of | Frequency of |
|---|---|---|---|---|---|
| Group | | animals | (mg/kg) | administration | administration |
| Vehicle | | 6 | — | i.v.. | 3 times |
| Paclitaxel | | 6 | 15 | i.v. | Once every 4 days |
| | | | | | for 4 times |
| MOCK control check | | 6 | 3 | i.v. | 3 times |
| LNP-siTUBB2-1092 | 1092 intravenous group | 6 | 3 | i.v. | 4 times |
| | 1092 intratumoral group | 6 | 3 | i.t. | 3 times |
| LNP-siTUBB2-457 | 457 intravenous group | 6 | 3 | i.v.. | 3 times |
| | 457 intratumoral group | 6 | 3 | i.t. | 3 times |

The results demonstrated that by 64 days after inoculation, only the 457 intravenous group of mice exhibited greater body weight loss than the paclitaxel group. Other groups of mice exhibited greater body weight loss than the vehicle group but less than the paclitaxel group. In terms of the tumor volume, the 1092 intravenous group and 457 intratumoral group exhibited smaller tumors compared with the paclitaxel group and other groups. In terms of the tumor inhibition index (TGI), the 1092 intravenous group and 457 intratumoral group maintained higher TGI values than the paclitaxel group even 20 days after administration cessation. At the experimental endpoint (64 days after inoculation and 27 days after administration cessation), their TGI values reached 42.28 and 39.13, respectively, whereas the TGI value of the paclitaxel group declined to 33.03. These indicate that LNP-siTUBB2-457 and 1092 can exert prolonged effects compared with paclitaxel, and can inhibit tumor growth.

The preferred embodiments of the present disclosure are described in detail above, which, however, are not intended to limit the present disclosure. Within the scope of the technical concept of the present disclosure, various simple modifications can be made to the technical solution of the present disclosure, all of which will fall within the protection scope of the present disclosure.

In addition, it should be noted that the various specific technical features described in the above specific embodiments can be combined in any suitable manner in which the features do not contradict each other. In order to avoid unnecessary repetition, such combinations will not be illustrated separately in the present disclosure.

1. An interfering RNA targeting a TUBB2 gene, wherein a target site sequence of the interfering RNA comprises the nucleotide sequence set forth in any one or two or more of SEQ ID NOs: 1-13.

2. The interfering RNA according to claim 1, comprising one or a combination of two or more of an siRNA, a dsRNA, an shRNA, an aiRNA, or an miRNA.

3. The interfering RNA according to claim 1, further comprising an over-hang.

4. The interfering RNA according to claim 3, wherein the over-hang is located at the 3′ terminus of a sense strand and/or an antisense strand of the interfering RNA.

5. The interfering RNA according to claim 3, wherein the over-hang is a deoxynucleoside.

6. The interfering RNA according to claim 1, comprising a sense strand and/or an antisense strand,
the sense strand comprises the nucleotide sequence set forth in any one or two or more of SEQ ID NOs: 14-26; and
the antisense strand comprises the nucleotide sequence set forth in any one or two or more of SEQ ID NOs: 27-39.

7. The interfering RNA according to claim 1, further comprising at least one modification, wherein the modification comprises a modification on a base, a sugar ring, and/or a phosphate backbone;
the modification on a base comprises, but is not limited to, a pyrimidine modification at position 5, a purine modification at position 8, a pseudouridine modification, and/or a 5-bromouracil substitution;
the modification on a sugar ring comprises, but is not limited to, a substitution of 2′-OH with H, OZ, Z, halo, SH, SZ, NH₂, NHZ, NZ₂, or CN group, Z being an alkyl group; and
the modification on a phosphate backbone comprises, but is not limited to, a phosphorothioate modification.

8. The interfering RNA according to claim 7, wherein the modification further comprises nucleotides having inosine, queuosine, xanthine, 2′-methylribose, a non-natural phosphodiester bond (e.g., methylphosphonate and phosphorothioate), and/or a peptide.

9. A delivery system of the interfering RNA according to claim 1, comprising the interfering RNA according to claim 1 and a vector.

10. The delivery system according to claim 9, wherein the vector is a viral vector or a non-viral vector;
the viral vector comprises one or a combination of two or more of a lentivirus vector, a retrovirus vector, an adenovirus vector, an adeno-associated virus vector, a poxvirus vector, or a herpesvirus vector; and
the non-viral vector comprises any one or a combination of two or more of a liposome, a lipid nanoparticle, a polymer, a polypeptide, an antibody, an aptamer, or N-acetylgalactosamine (GalNAc).

11. The delivery system according to claim 10, wherein the lipid nanoparticle/liposome comprises one or a combination of two or more of a cationic lipid, a neutral lipid, a polyethylene glycol lipid, a steroidal lipid, or an anionic lipid.

12. The delivery system according to claim 11, wherein the cationic lipid comprises one or a combination of two or more of stearamide (SA), lauryltrimethylammonium bromide, hexadecyltrimethylammonium bromide, myristyltrimethylammonium bromide, dimethyldioctadecylammonium bromide (DDAB), [(4-hydroxybutyl)azanediyl]bis(hexane-6,1-diyl)bis(2-hexyldecanoate) (ALC-0315), 1,2-dioleoyloxy-3-(trimethylammonium)propane (DOTAP), 1,2-di-(9Z-octadecenoyl)-3-trimethylammonium-propane and 1,2-dihexadecanoyl-3-trimethylammonium-propane, 3β-[N—(N′,N′-dimethylaminoethane)-carbamoyl]cholesterol (DC cholesterol), dimethyldioctadecylammonium (DDA), 1,2-dimyristoyl-3-trimethylammonium propane (DMTAP), dipalmitoyl(C16:0)trimethylammonium propane (DPTAP), distearoyltrimethylammonium propane (DSTAP), N-[1-(2,3-diallyloxy)propyl]-N,N,N-trimethylammonium chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium propane (DODAP), 1,2-dilinoleyloxy-3-dimethylaminopropane (DLinDMA), 1,2-ditetradecanoyl-3-dimethylammonium-propane, 1,2-dihexadecanoyl-3-dimethylammonium-propane and 1,2-dioctadecanoyl-3-dimethylammonium-propane, 1,2-dioleoyl-c-(4′-trimethylammonium)-butanoyl-sn-glycerol (DOTB), dioctadecylamidoalanyl spermine, SAINT-2, polycationic lipid 2,3-dioleoyloxy-N-[2(spermine-carboxamido)ethyl]-N,N-dimethyl-1-propanaminium trifluoroacetate (DOSPA),

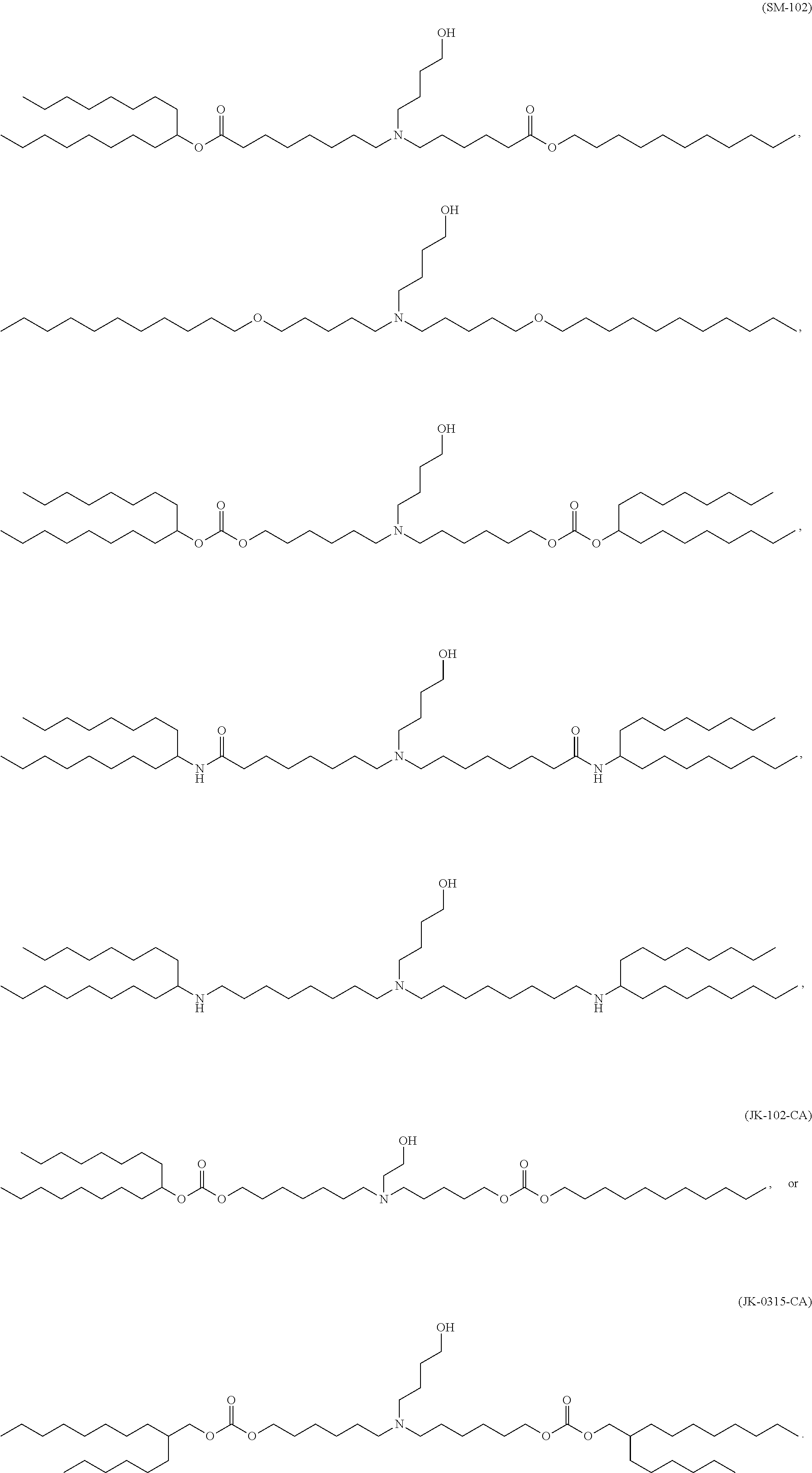

13. The delivery system according to claim 11, wherein the neutral lipid comprises one or a combination of two or more of 1,2-distearoyl-sn-glycero-3-phosphocholine (DSPC), 1,2-dipalmitoyl-sn-glycero-3-phosphocholine (DPPC), 1,2-dioleoyl-sn-glycero-3-phosphoethanolamine (DOPE), 1,2-dipalmitoyl-sn-glycero-3-phosphoethanolamine (DPPE), 1,2-dimyristoyl-sn-glycero-3-phosphoethanolamine (DMPE), 2-dioleoyl-sn-glycero-3-phospho-(1′-rac-glycerol) (DOPG), oleoyl phosphatidylcholine (POPC), 1-palmitoyl-2-oleoyl phosphatidylethanolamine (POPE), or distearoylphosphatidylethanolamine (DSPE).

14. The delivery system according to claim 11, wherein the polyethylene glycol lipid comprises one or a combination of two or more of 2-[(polyethylene glycol)-2000]-N,N-ditetradecylacetamide (ALC-0159), 1,2-dimyristoyl-sn-glyceromethoxypolyethylene glycol (PEG-DMG), 1,2-distearoyl-sn-glycero-3-phosphoethanolamine-N-[amino(polyethylene glycol)](PEG-DSPE), PEG-disterol glycerol (PEG-DSG), PEG-dipalmitoyl, PEG-dioleyl, PEG-distearyl, PEG-diacylglycerol amide (PEG-DAG), PEG-dipalmitoyl phosphatidylethanolamine (PEG-DPPE) or PEG-1,2-dimyristoylacyloxypropyl-3-amine (PEG-c-DMA), 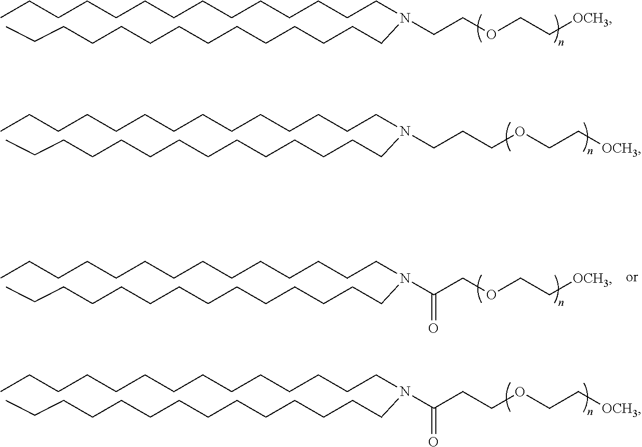
wherein n is selected from integers of 20-300.

15. The delivery system according to claim 11, wherein the polyethylene glycol lipid is a polyethylene glycol lipid with a single molecular weight,
the polyethylene glycol lipid comprises

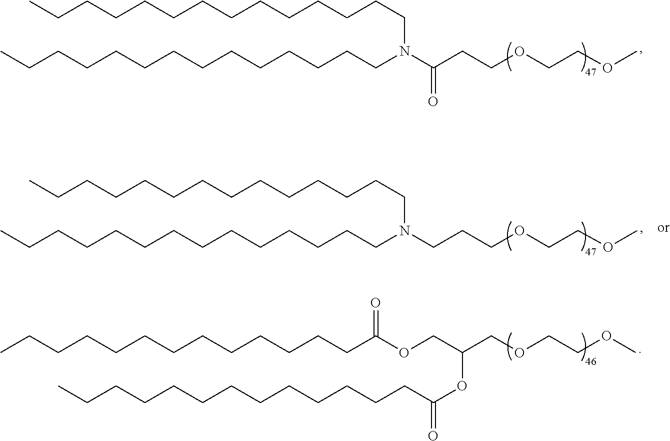

16. The delivery system according to claim 11, wherein the cationic lipid is a steroid-cationic lipid compound,
and the compound has a structure of one or two or more of

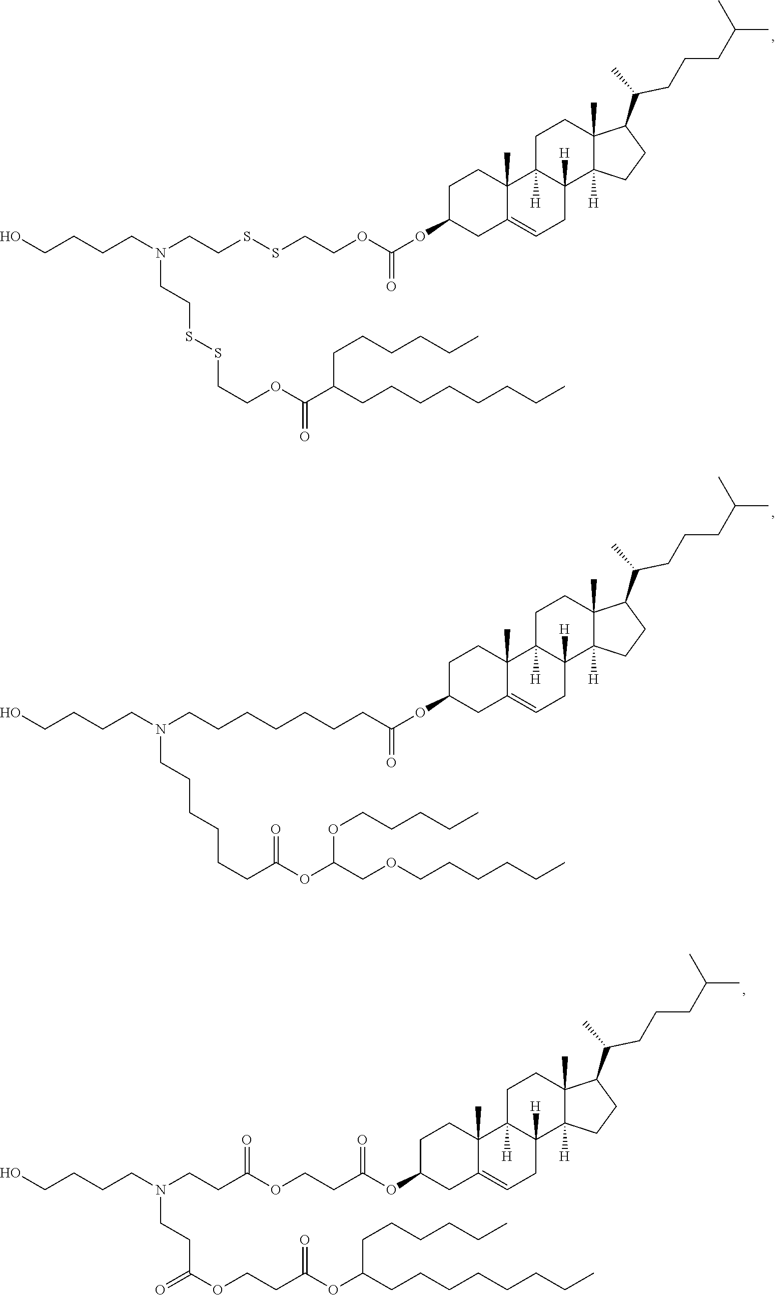

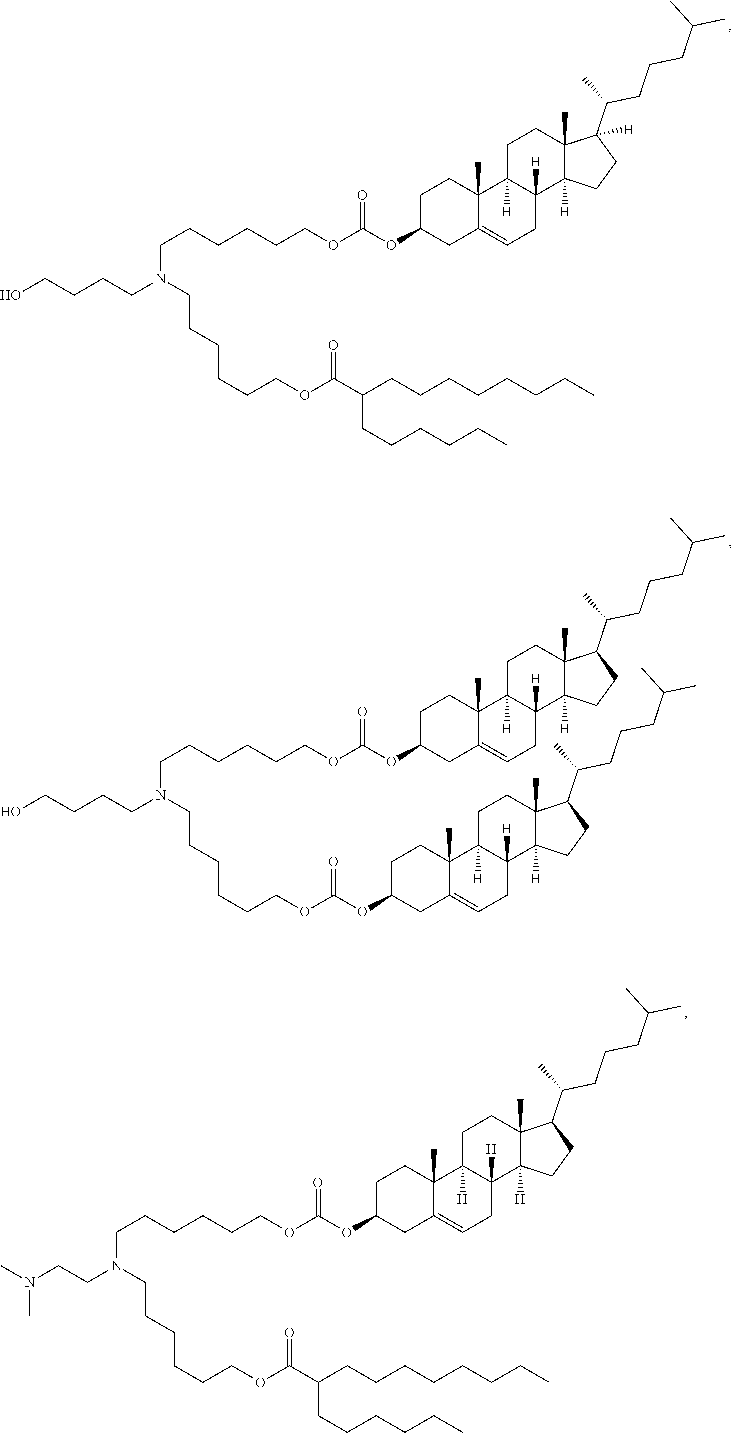

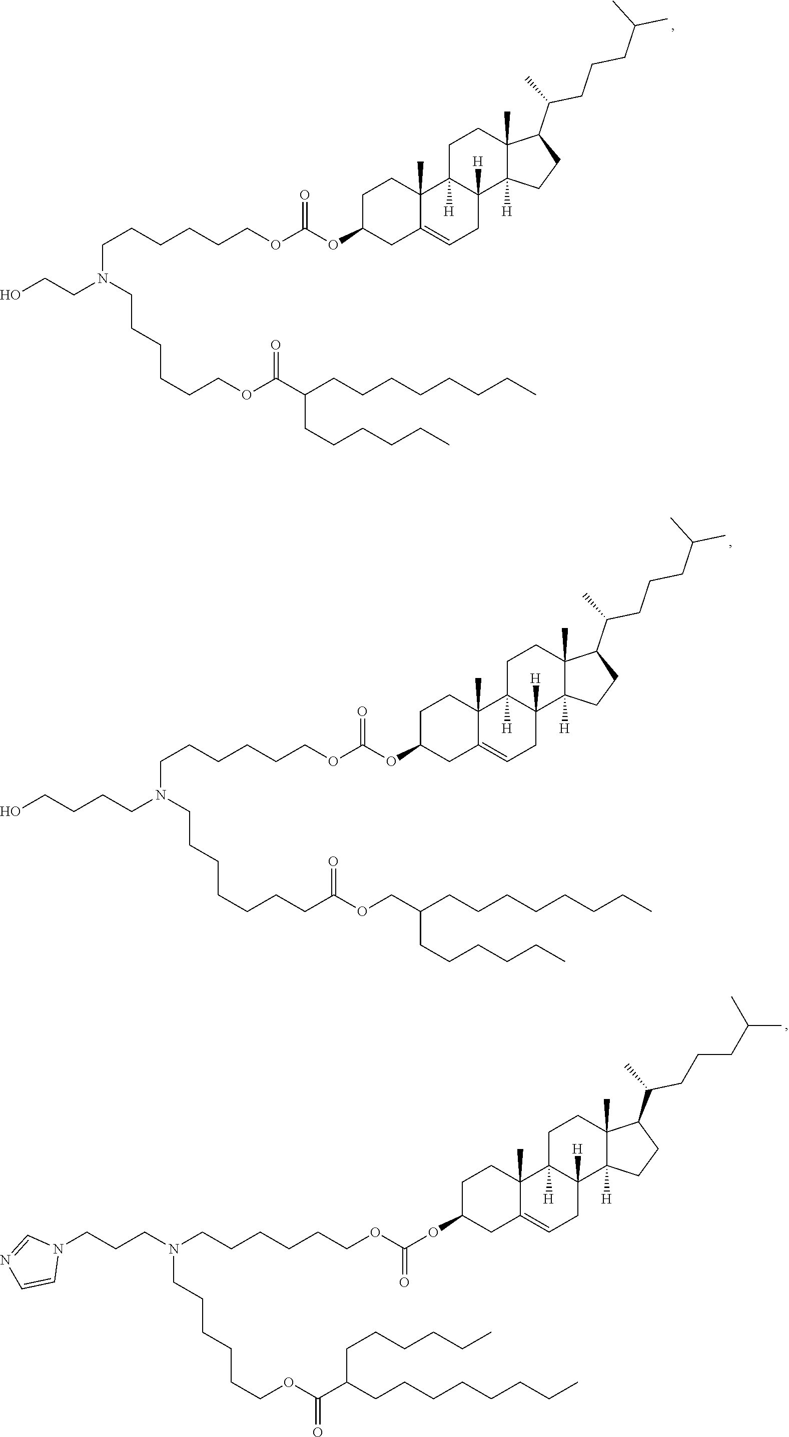

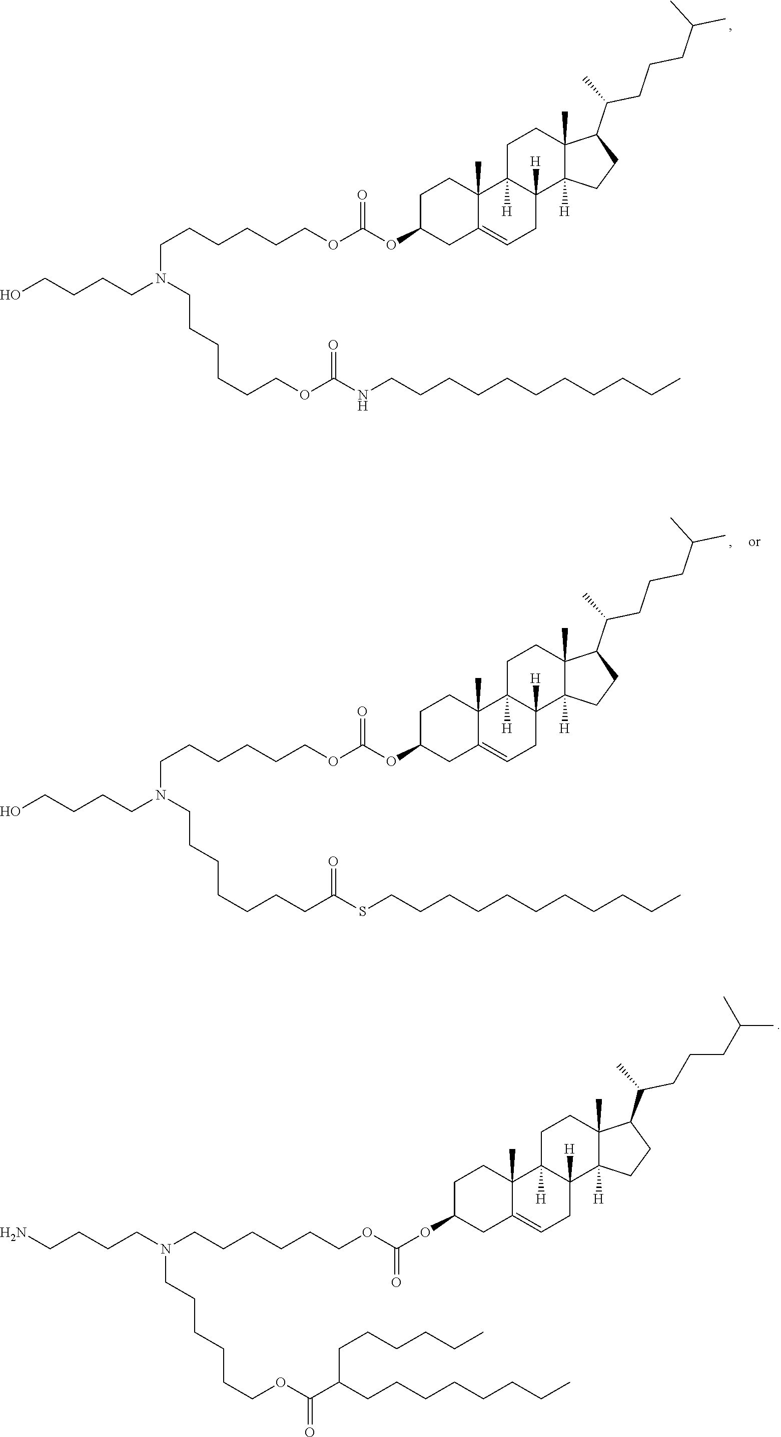

17. The delivery system according to claim 11, wherein the anionic liposome comprises one or a combination of two or more of dioleoyl phosphatidylglycerol or dioleoyl phosphatidylethanolamine.

18. The delivery system according to claim 11, wherein the steroidal lipid comprises one or a combination of two or more of avenasterol, β-sitosterol, brassicasterol, ergocalciferol, campesterol, cholestanol, cholesterol, coprostanol, dehydrocholesterol, desmosterol, dihydroergocalciferol, dihydrocholesterol, dihydroergosterol, dinosterol, epicholesterol, ergosterol, fucosterol, hexahydrolumisterol, hydroxycholesterol, lanosterol, lumisterol, saringosterol, sitostanol, sitosterol, stigmastanol, stigmasterol, cholic acid, glycocholic acid, taurocholic acid, deoxycholic acid, or lithocholic acid.

19.-32. (canceled)

33. A method for inhibiting TUBB2 expression, comprising adding the interfering RNA according to claim 1, the delivery system comprising the interfering RNA thereof.

34. A method for treating a disease related to an increase of TUBB2 expression or excessive proliferation of cells, comprising administering to a subject a therapeutically effective amount of the interfering RNA according to claim 1, the delivery system comprising the interfering RNA thereof.

35.-37. (canceled)


SEQUENCE LISTING (63 sequences)

SEQ ID NO: 1 - DNA, 19 bp, synthetic construct
  source 1..19: mol_type = other DNA
tgcgtgagatcgtgcacat

SEQ ID NO: 2 - DNA, 19 bp, synthetic construct
  source 1..19: mol_type = other DNA
ggtcatcagtgatgagcat

SEQ ID NO: 3 - DNA, 19 bp, synthetic construct
  source 1..19: mol_type = other DNA
gtgatgagcatgggattga

SEQ ID NO: 4 - DNA, 19 bp, synthetic construct
  source 1..19: mol_type = other DNA
gaccattcggccagatctt

SEQ ID NO: 5 - DNA, 19 bp, synthetic construct
  source 1..19: mol_type = other DNA
tcggttaggtctggaccat

SEQ ID NO: 6 - DNA, 19 bp, synthetic construct
  source 1..19: mol_type = other DNA
agatcttcagaccagacaa

SEQ ID NO: 7 - DNA, 19 bp, synthetic construct
  source 1..19: mol_type = other DNA
acaatttcgtgtttggcca

SEQ ID NO: 8 - DNA, 19 bp, synthetic construct
  source 1..19: mol_type = other DNA
tcctggatgtggtgaggaa

SEQ ID NO: 9 - DNA, 19 bp, synthetic construct
  source 1..19: mol_type = other DNA
tggatgtggtgaggaagga

SEQ ID NO: 10 - DNA, 25 bp, synthetic construct
  source 1..25: mol_type = other DNA
gagcagatgctcaacgtgcagaaca

SEQ ID NO: 11 - DNA, 25 bp, synthetic construct
  source 1..25: mol_type = other DNA
agcagatgctcaacgtgcagaacaa

SEQ ID NO: 12 - DNA, 19 bp, synthetic construct
  source 1..19: mol_type = other DNA
ccgagagcaacatgaacga

SEQ ID NO: 13 - DNA, 19 bp, synthetic construct
  source 1..19: mol_type = other DNA
agagcaacatgaacgacct

SEQ ID NO: 14 - RNA, 19 nt, synthetic construct
  source 1..19: mol_type = other RNA
tgcgtgagatcgtgcacat

SEQ ID NO: 15 - RNA, 19 nt, synthetic construct
  source 1..19: mol_type = other RNA
ggtcatcagtgatgagcat

SEQ ID NO: 16 - RNA, 19 nt, synthetic construct
  source 1..19: mol_type = other RNA
gtgatgagcatgggattga

SEQ ID NO: 17 - RNA, 19 nt, synthetic construct
  source 1..19: mol_type = other RNA
gaccattcggccagatctt

SEQ ID NO: 18 - RNA, 19 nt, synthetic construct
  source 1..19: mol_type = other RNA
tcggttaggtctggaccat

SEQ ID NO: 19 - RNA, 19 nt, synthetic construct
  source 1..19: mol_type = other RNA
agatcttcagaccagacaa

SEQ ID NO: 20 - RNA, 19 nt, synthetic construct
  source 1..19: mol_type = other RNA
acaatttcgtgtttggcca

SEQ ID NO: 21 - RNA, 19 nt, synthetic construct
  source 1..19: mol_type = other RNA
tcctggatgtggtgaggaa

SEQ ID NO: 22 - RNA, 19 nt, synthetic construct
  source 1..19: mol_type = other RNA
tggatgtggtgaggaagga

SEQ ID NO: 23 - RNA, 25 nt, synthetic construct
  source 1..25: mol_type = other RNA
gagcagatgctcaacgtgcagaaca

SEQ ID NO: 24 - RNA, 25 nt, synthetic construct
  source 1..25: mol_type = other RNA
agcagatgctcaacgtgcagaacaa

SEQ ID NO: 25 - RNA, 19 nt, synthetic construct
  source 1..19: mol_type = other RNA
ccgagagcaacatgaacga

SEQ ID NO: 26 - RNA, 19 nt, synthetic construct
  source 1..19: mol_type = other RNA
agagcaacatgaacgacct

SEQ ID NO: 27 - RNA, 19 nt, synthetic construct
  source 1..19: mol_type = other RNA
atgtgcacgatctcacgca

SEQ ID NO: 28 - RNA, 19 nt, synthetic construct
  source 1..19: mol_type = other RNA
atgctcatcactgatgacc

SEQ ID NO: 29 - RNA, 19 nt, synthetic construct
  source 1..19: mol_type = other RNA
tcaatcccatgctcatcac

SEQ ID NO: 30 - RNA, 19 nt, synthetic construct
  source 1..19: mol_type = other RNA
aagatctggccgaatggtc

SEQ ID NO: 31 - RNA, 19 nt, synthetic construct
  source 1..19: mol_type = other RNA
atggtccagacctaaccga

SEQ ID NO: 32 - RNA, 19 nt, synthetic construct
  source 1..19: mol_type = other RNA
ttgtctggtctgaagatct

SEQ ID NO: 33 - RNA, 19 nt, synthetic construct
  source 1..19: mol_type = other RNA
tggccaaacacgaaattgt

SEQ ID NO: 34 - RNA, 19 nt, synthetic construct
  source 1..19: mol_type = other RNA
ttcctcaccacatccagga

SEQ ID NO: 35 - RNA, 19 nt, synthetic construct
  source 1..19: mol_type = other RNA
tccttcctcaccacatcca

SEQ ID NO: 36 - RNA, 25 nt, synthetic construct
  source 1..25: mol_type = other RNA
tgttctgcacgttgagcatctgctc

SEQ ID NO: 37 - RNA, 25 nt, synthetic construct
  source 1..25: mol_type = other RNA
ttgttctgcacgttgagcatctgct

SEQ ID NO: 38 - RNA, 19 nt, synthetic construct
  source 1..19: mol_type = other RNA
tcgttcatgttgctctcgg

SEQ ID NO: 39 - RNA, 19 nt, synthetic construct
  source 1..19: mol_type = other RNA
aggtcgttcatgttgctct

SEQ ID NO: 40 - RNA, 21 nt, synthetic construct
  source 1..21: mol_type = other RNA
ttctccgaacgtgtcacgttt

SEQ ID NO: 41 - RNA, 21 nt, synthetic construct
  source 1..21: mol_type = other RNA
acgtgacacgttcggagaatt

SEQ ID NO: 42 - RNA, 19 nt, synthetic construct
  source 1..19: mol_type = other RNA
tcatgaacaccttcagcgt

SEQ ID NO: 43 - RNA, 19 nt, synthetic construct
  source 1..19: mol_type = other RNA
agcagatgttcgactccaa

SEQ ID NO: 44 - RNA, 19 nt, synthetic construct
  source 1..19: mol_type = other RNA
cagatgttcgactccaaga

SEQ ID NO: 45 - RNA, 19 nt, synthetic construct
  source 1..19: mol_type = other RNA
agatgttcgactccaagaa

SEQ ID NO: 46 - RNA, 19 nt, synthetic construct
  source 1..19: mol_type = other RNA
acgctgaaggtgttcatga

SEQ ID NO: 47 - RNA, 19 nt, synthetic construct
  source 1..19: mol_type = other RNA
ttggagtcgaacatctgct

SEQ ID NO: 48 - RNA, 19 nt, synthetic construct
  source 1..19: mol_type = other RNA
tcttggagtcgaacatctg

SEQ ID NO: 49 - RNA, 19 nt, synthetic construct
  source 1..19: mol_type = other RNA
ttcttggagtcgaacatct

SEQ ID NO: 50 - DNA, 20 bp, synthetic construct
  source 1..20: mol_type = other DNA
gccagatcttcagaccagac

SEQ ID NO: 51 - DNA, 20 bp, synthetic construct
  source 1..20: mol_type = other DNA
actccttcctcaccacatcc

SEQ ID NO: 52 - DNA, 21 bp, synthetic construct
  source 1..21: mol_type = other DNA
ggcacgatggattcggttagg

SEQ ID NO: 53 - DNA, 23 bp, synthetic construct
  source 1..23: mol_type = other DNA
acacgaaattgtctggtctgaag

SEQ ID NO: 54 - DNA, 23 bp, synthetic construct
  source 1..23: mol_type = other DNA
catgagaagtatgacaacagcct

SEQ ID NO: 55 - DNA, 22 bp, synthetic construct
  source 1..22: mol_type = other DNA
agtccttccacgataccaaagt

SEQ ID NO: 56 - RNA, 21 nt, synthetic construct
  source 1..21: mol_type = other RNA
  modified_base 20..21: mod_base = OTHER; note = thymine
  modified_base 1..4: mod_base = OTHER; note = 2-O-methyl
  modified_base 8: mod_base = OTHER; note = 2-O-methyl
  modified_base 10: mod_base = OTHER; note = 2-O-methyl
  modified_base 13: mod_base = OTHER; note = 2-O-methyl
tcctggatgtggtgaggaatt

SEQ ID NO: 57 - RNA, 21 nt, synthetic construct
  source 1..21: mol_type = other RNA
  modified_base 20..21: mod_base = OTHER; note = thymine
  modified_base 1: mod_base = p
  modified_base 4: mod_base = p
  modified_base 8: mod_base = p
  modified_base 10: mod_base = p
  modified_base 13: mod_base = p
nccnggangnggngaggaatt

SEQ ID NO: 58 - RNA, 25 nt, synthetic construct
  source 1..25: mol_type = other RNA
  modified_base 8: mod_base = p
  modified_base 11: mod_base = p
  modified_base 17: mod_base = p
gagcagangcncaacgngcagaaca

SEQ ID NO: 59 - RNA, 25 nt, synthetic construct
  source 1..25: mol_type = other RNA
  modified_base 1..3: mod_base = OTHER; note = 2-O-methyl
  modified_base 4: mod_base = OTHER; note = 2-deoxy-2-fluoro
  modified_base 5: mod_base = OTHER; note = 2-O-methyl
  modified_base 6: mod_base = OTHER; note = 2-deoxy-2-fluoro
  modified_base 7: mod_base = OTHER; note = 2-O-methyl
  modified_base 8: mod_base = OTHER; note = 2-deoxy-2-fluoro
  modified_base 9: mod_base = OTHER; note = 2-O-methyl
  modified_base 10..12: mod_base = OTHER; note = 2-deoxy-2-fluoro
  modified_base 13: mod_base = OTHER; note = 2-O-methyl
  modified_base 14: mod_base = OTHER; note = 2-deoxy-2-fluoro
  modified_base 15: mod_base = OTHER; note = 2-O-methyl
  modified_base 16: mod_base = OTHER; note = 2-deoxy-2-fluoro
  modified_base 17: mod_base = OTHER; note = 2-O-methyl
  modified_base 18: mod_base = OTHER; note = 2-deoxy-2-fluoro
  modified_base 19: mod_base = OTHER; note = 2-O-methyl
  modified_base 20: mod_base = OTHER; note = 2-deoxy-2-fluoro
  modified_base 21: mod_base = OTHER; note = 2-O-methyl
  modified_base 22: mod_base = OTHER; note = 2-deoxy-2-fluoro
  modified_base 23..25: mod_base = OTHER; note = 2-O-methyl
  modified_base 1^2: mod_base = OTHER; note = Phosphorothioate
  modified_base 2^3: mod_base = OTHER; note = Phosphorothioate
gagcagatgctcaacgtgcagaaca

SEQ ID NO: 60 - RNA, 21 nt, synthetic construct
  source 1..21: mol_type = other RNA
  modified_base 20..21: mod_base = OTHER; note = thymine
  modified_base 1..6: mod_base = OTHER; note = 2-O-methyl
  modified_base 8..9: mod_base = OTHER; note = 2-O-methyl
  modified_base 11: mod_base = OTHER; note = 2-O-methyl
  modified_base 13..15: mod_base = OTHER; note = 2-O-methyl
ttcctcaccacatccaggatt

SEQ ID NO: 61 - RNA, 21 nt, synthetic construct
  source 1..21: mol_type = other RNA
  modified_base 20..21: mod_base = OTHER; note = thymine
  modified_base 1..2: mod_base = p
  modified_base 5: mod_base = p
  modified_base 13: mod_base = p
nnccncaccacanccaggatt

SEQ ID NO: 62 - RNA, 25 nt, synthetic construct
  source 1..25: mol_type = other RNA
  modified_base 1: mod_base = p
  modified_base 3..4: mod_base = p
  modified_base 6: mod_base = p
  modified_base 12..13: mod_base = p
  modified_base 19: mod_base = p
  modified_base 21: mod_base = p
  modified_base 24: mod_base = p
ngnncngcacgnngagcancngcnc

SEQ ID NO: 63 - RNA, 25 nt, synthetic construct
  source 1..25: mol_type = other RNA
  modified_base 1..3: mod_base = OTHER; note = 2-deoxy-2-fluoro
  modified_base 5: mod_base = OTHER; note = 2-deoxy-2-fluoro
  modified_base 7: mod_base = OTHER; note = 2-deoxy-2-fluoro
  modified_base 9: mod_base = OTHER; note = 2-deoxy-2-fluoro
  modified_base 13: mod_base = OTHER; note = 2-deoxy-2-fluoro
  modified_base 15: mod_base = OTHER; note = 2-deoxy-2-fluoro
  modified_base 17: mod_base = OTHER; note = 2-deoxy-2-fluoro
  modified_base 19: mod_base = OTHER; note = 2-deoxy-2-fluoro
  modified_base 21: mod_base = OTHER; note = 2-deoxy-2-fluoro
  modified_base 23..25: mod_base = OTHER; note = 2-deoxy-2-fluoro
  modified_base 4: mod_base = OTHER; note = 2-O-methyl
  modified_base 6: mod_base = OTHER; note = 2-O-methyl
  modified_base 8: mod_base = OTHER; note = 2-O-methyl
  modified_base 10..12: mod_base = OTHER; note = 2-O-methyl
  modified_base 14: mod_base = OTHER; note = 2-O-methyl
  modified_base 16: mod_base = OTHER; note = 2-O-methyl
  modified_base 18: mod_base = OTHER; note = 2-O-methyl
  modified_base 20: mod_base = OTHER; note = 2-O-methyl
  modified_base 22: mod_base = OTHER; note = 2-O-methyl
  modified_base 1^2: mod_base = OTHER; note = Phosphorothioate
  modified_base 2^3: mod_base = OTHER; note = Phosphorothioate
  modified_base 23^24: mod_base = OTHER; note = Phosphorothioate
  modified_base 24^25: mod_base = OTHER; note = Phosphorothioate
tgttctgcacgttgagcatctgctc